(19) United States
(12) Patent Application Publication
(10) Pub. No.: US 2002/0193566 A1
(43) Pub. Date: Dec. 19, 2002

(54) Thermodynamic propensities of amino acids in the native state ensemble: implications for fold recognition

(76) Inventors: Vince Hilser, Galveston, TX (US); Robert O. Fox, Galveston, TX (US)

Correspondence Address: FULBRIGHT & JAWORSKI, LLP, 1301 MCKINNEY, SUITE 5100, HOUSTON, TX, 77010-3095, US

(21) Appl. No.: 10/047,724

(22) Filed: Jan. 15, 2002

Related U.S. Application Data
(60) Provisional application No. 60/261,733, filed on Jan. 16, 2001.

(51) Int. Cl. G06G 7/48; G06G 7/58; G06F 19/00; C07K 14/00

(52) U.S. Cl. 530/350; 702/19; 703/11

(57) ABSTRACT

The present invention relates to a system and computer-based method that is used to determine thermodynamic environment differences within a protein. This method is used to construct a database of proteins, wherein the database can be used to identify correct sequences that correspond to a particular target fold.

This Applications claims priority to U.S. Provisional Application No. 60/261,733, which was filed on Jan. 16, 2001.

The work herein was supported by grants from the United States Government. The United States Government may have certain rights in the invention.

### BACKGROUND OF THE INVENTION

I. Field of the Invention

The present invention relates to the field of structural biology. More particularly, the present invention relates to a protein database and methods of developing a protein database that contains all of the thermodynamic information necessary to encode a three-dimensional protein structure.

II. Related Art

It is a longstanding idea that protein structures are the result of an amino acid chain finding its global free energy minimum in the solvent environment (Anfinsen, 1973). Several exceptions to this so-called “thermodynamic control” have been discovered in recent years, including examples of proteins whose folding may be under “kinetic control” (Baker et al., 1992, Cohen, 1999) and proteins requiring information not completely contained in the amino acid sequence (e.g., chaperone-assisted folding (Feldman & Frydman 2000, Fink 1999)). Although thermodynamic control is widely accepted as the default behavior for correct folding (Jackson, 1998), a detailed understanding of the forces involved in thermodynamic control and how atomic interactions relate amino acid sequence to the folding and stability of the native structure has still proven elusive.

Despite the progress that has been made in protein folding, obstacles have prevented an accurate structure prediction algorithm. An obstacle in developing an accurate structure prediction algorithm has been the lack of suitable potentials for calculating the free energies of different conformations of a given protein molecule. In 1992, high-pressure liquid chromatography (HPLC) was used to quantitate the energies of pairwise interactions between amino acid side chains (Pochapsky and Gopen, 1992). Yet further, in 1999, Pochapsky used HPLC to further study the thermodynamic interactions between amino acid side chains. A stationary phase was prepared for use in an HPLC. The phase was prepared by derivatizing microparticulate silica gels with functionality mimicking the side chain of hydrophobic and amphiphilic amino acid analytes (Pereira de Araujo et al., 1999). Thus, this variation of an HPLC method compares entropies and free energies of interaction using different derivatized microparticulate silica gels.

The present invention uses a computer-based algorithm to address for the first time whether amino acid residue types have distinct preferences for thermodynamic environments in the folded native structure of a protein, and whether a scoring matrix based solely on thermodynamic information (independent of explicit structural constraints) can be used to identify correct sequences that correspond to a particular target fold. This is done by means of a unique approach in which the regional stability differences within a protein are determined for a database of proteins using the COREX algorithm (Hilser & Freire, 1996). The COREX algorithm generates an ensemble of states using the high-resolution structure as a template. Based on the relative probability of the different states in the ensemble, different regions of the protein are found to be more stable than others. Thus, the COREX algorithm provides access to residue-specific free energies of folding.

### BRIEF SUMMARY OF THE INVENTION

One embodiment of the present invention is directed to a system and method of developing a protein database that contains all of the thermodynamic information necessary to encode a three-dimensional protein structure

Another embodiment of the present invention comprises a protein database comprising nonhomologus proteins having known residue-specific free energies of folding of the proteins. In specific embodiments, the database comprises globular proteins.

In further embodiments, the database is determined by a computational method comprising the step of determining a stability constant from the ratio of the summed probability of all states in the ensemble in which a residue j is in a folded conformation to the summed probability of all states in which j is in an unfolded conformation according to the equation,

$${K}_{f , j} = \frac{\sum {P}_{f , j}}{\sum {P}_{nf , j}}$$

Another specific embodiment of the present invention comprises that the stability constants for the residues are arranged into at least one of the three thermodynamic classification groups selected from the group consisting of stability, enthalpy, and entropy.

In specific embodiments, the stability thermodynamic classification group comprises high stability, medium stability and low stability. More particularly, the residues in the high stability classification comprises phenylalanine, tryptophan and tyrosine. The residues in the low stability classification comprises glycine and proline. And the residues in the medium stability classification comprises asparagine and glutamic acid.

Yet further, the enthalpy thermodynamic classification group comprises high enthalpy and low enthalpy. Enthalpy comprises a ratio of the contributions of polar and apolar components.

In another specific embodiment, the entropy thermodynamic classification group comprises high entropy and low entropy. Entropy comprises a ratio of the contributions of polar and apolar components.

In a further embodiment, the stability constants for the residues are arranged into twelve thermodynamic classifications selected from the group consisting of HHH, MHH, LHH, HHL, MHL, LHL, HLL, MLL, LLL, HLH, MLH and LLH.

Another embodiment of the present invention is a method of developing a protein database comprising the steps of: inputting high resolution structures of proteins; generating an ensemble of incrementally different conformational states by combinatorial unfolding of a set of predefined folding units in all possible combinations of each protein; determining the probability of each said conformational state; calculating a residue-specific free energy of each said conformational state; and classifying a stability constant into at least one thermodynamic classification group selected from the group consisting of stability, enthalpy, and entropy. Specifically, the protein database comprises globular and nonhomologous proteins.

In specific embodiments, the generating step comprises dividing the proteins into folding units by placing a block of windows over the entire sequence of the protein and sliding the block of windows one residue at a time.

In further specific embodiment, the determining step comprises determining the free energy of each of the conformational states in the ensemble; determining the Boltzmann weight [Kᵢ=exp(−ΔGᵢ/RT)] of each state; and determining the probability of each state using the equation:

$${P}_{i} = \frac{{K}_{i}}{\sum {K}_{i}^{′}}$$

In specific embodiments, the calculating step comprises determining the energy difference between all microscopic states in which a particular residue is folded and all such states in which it is unfolded using the equation

ΔG_{f,j}=−RT·lnκ_{f,j}

Another embodiment of the present invention is a method of identifying a protein fold comprising determining the distribution of amino acid residues in different thermodynamic environments corresponding to a known protein structure. Specifically, determining the distribution of amino acid residues comprises constructing scoring matrices derived of thermodynamic information. The scoring matrices are derived from COREX thermodynamic information selected from the group consisting of stability, enthalpy, and entropy.

The aforementioned embodiments of the present invention may be readily implemented as a computer-based system. One embodiment of such a computer-based system includes a computer program that receives an input of high resolution structure data for one or more proteins. The computer-based program utilizes this data to determine the amino acid thermodynamic classifications for the proteins. These amino acid thermodynamic classifications may then be stored in a database. The database of the system preferably has a data structure with a field or fields for storing a value for an amino acid name or amino acid abbreviation, and one or more classification fields for storing a numerical value for a thermodynamic classification for a particular amino acid. Additionally, this data structure may have a field for storing a value representing the summed total of each of the numerical values for each thermodynamic classification for a particular amino acid.

In one embodiment of the inventive system, the computer-based program performs a process to generate thermodynamic classifications for a protein which includes inputting high resolution structures of proteins, generating an ensemble of incrementally different conformational states by combinatorial unfolding of a set of predefined folding units in all possible combinations of each protein, determining the probability of each said conformational state, calculating a residue-specific free energy of each said conformational state, and classifying a stability constant into a thermodynamic classification group. Additionally, the computer-based program may have a probability determination module to determine the free energy of each of the conformational states in a computed ensemble, determine a Boltzmann weight, and then determine the probability of each state.

Moreover, the computer-based program of the inventive system may have a display/reporting module for producing one or more graphical reports to a screen or a print-out. Some of these reports include: a display of a three-dimensional protein structure based on said amino acid thermodynamic classifications; a scatter-plot of normalized frequencies of COREX stability data versus normalized frequencies of average side chain surface exposure; and a chart displaying thermodynamic environments for amino acids of a protein.

Another aspect of the inventive methods is that they may be stored as computer executable instructions on computer-readable medium.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A and FIG. 1B are a schematic description of the COREX algorithm applied to the crystal structure of the ovomucoid third domain, OM3 (2ovo). FIG. 1A summarizes the partitioning strategy of the COREX algorithm. FIG. 1B illustrates the solvent exposed surface area (ASA) contributing to the energetics of microstate 32.

FIG. 2 is a comparison of hydrogen exchange protection factors predicted from COREX data with experimental values for ovomucoid third domain (2ovo). Unfilled vertical bars denote predicted values, and filled vertical bars denote experimental values (Swint-Kruse & Robertson, 1996). The solid line denotes lnκf values. The simulated temperature of the COREX calculation was set at 30° C. to match the experimental conditions. Secondary structure is given by labeled horizontal lines. Asterisks show the positions of Thr 47 and Thr 49, referred to in the text.

FIG. 3A, FIG. 3B, FIG. 3C, FIG. 3D, FIG. 3E, FIG. 3F, FIG. 3G, FIG. 3H, FIG. 3I, FIG. 3J, FIG. 3K, FIG. 3L, FIG. 3M, FIG. 3N, FIG. 3N, FIG. 3O, FIG. 3P, FIG. 3Q, FIG. 3R, FIG. 3S and FIG. 3T comprise normalized frequencies of COREX stability data as a function of amino acid type. FIG. 3A shows the data as a function of the amino acid alanine. FIG. 3B shows the data as a function of the amino acid arginine. FIG. 3C shows the data as a function of the amino acid asparagine. FIG. 3D shows the data as a function of the amino acid aspartic acid. FIG. 3E shows the data as a function of the amino acid cysteine. FIG. 3F shows the data as a function of the amino acid glutamine. FIG. 3G shows the data as a function of the amino acid glutamic acid. FIG. 3H shows the data as a function of the amino acid glycine. FIG. 3I shows the data as a function of the amino acid histidine. FIG. 3J shows the data as a function of the amino acid isoleucine. FIG. 3K shows the data as a function of the amino acid leucine. FIG. 3L shows the data as a function of the amino acid lysine. FIG. 3M shows the data as a function of the amino acid methionine. FIG. 3N shows the data as a function of the amino acid phenylalanine. FIG. 3O shows the data as a function of the amino acid proline. FIG. 3P shows the data as a function of the amino acid serine. FIG. 3Q shows the data as a function of the amino acid threonine. FIG. 3R shows the data as a function of the amino acid tryptophan. FIG. 3S shows the data as a function of the amino acid tyrosine. FIG. 3T shows the data as a function of the amino acid valine. In each histogram, the low stability bin is on the left, the medium stability bin is in the middle, and the high stability bin is on the right. The data used in each histogram was taken from the 2922 residue data set, as given in Table 2.

FIG. 4 is a scatterplot of normalized frequencies of COREX stability data versus normalized frequencies of average side chain surface area exposure. Average side chain exposure in the native structure was calculated by using a moving window of five residues, similar to the basis of the COREX algorithm. These values were then binned into high, medium, and low surface area exposure.

FIG. 5A, FIG. 5B, FIG. 5C and FIG. 5D illustrate a summary of fold-recognition results for COREX stability and DSSP secondary structure scoring matrices for 44 targets. Black bars denote real data (either inκf or secondary structure), and striped bars denote the average of three random data sets. FIG. 5A shows the lnκf scoring matrix local alignment algorithm. FIG. 5B shows the inκf scoring matrix global alignment algorithm. FIG. 5C shows the secondary structure scoring matrix local alignment algorithm. FIG. 5D shows the secondary structure scoring matrix global alignment algorithm.

FIG. 6A, FIG. 6B and FIG. 6C illustrate examples of successful local alignment for three targets. Results for target 1igd (Protein G) are shown in FIG. 6A, results for target 1vcc (DNA topoisomerase I) are shown in FIG. 6B, and results for target 2ait (tendamistat) are shown in FIG. 6C. The thin black line represents COREX calculated stability data (lnκf) for the protein target. The filled circles connected by a thick black line correspond to the cumulative matrix score contributed by each residue. Scores that did not contribute to the final score due to the rules of the local alignment algorithm (Smith & Waterman, 1981) are shown as unfilled circles connected by a thick dashed line.

FIG. 7 is a correlation between stability data derived from the database of 44 proteins used in this work and stability data derived from an independent database of 50 proteins. Data on the x-axis are taken from the normalized histograms in FIG. 3A-FIG. 3T. Data on the y-axis are derived from an identical COREX analysis of an independent database of 3304 residues from 50 PDB structures not contained in the original database. Open circles denote the values for His, a residue type with low statistics in both databases. The dashed line represents a perfect correlation.

FIG. 8A and FIG. 8B illustrate the results of a COREX calculation for the bacterial cold-shock protein cspA (PDB 1mjc). FIG. 8A shows a plot of calculated thermodynamic stability, lnκ_{f,j}, as a function of residue number for cspA. The simulated temperature was 25.0° C. Regions of relatively high, medium, and low stability, are shown in dark gray, light gray, and black, respectively. Secondary structure elements, as defined by the program DSSP, (Kabsch and Sander, 1983) are labeled. FIG. 8B locates the relative calculated stabilities of each residue in the 1mjc crystal structure. Note that a given secondary structural element is predicted to have varying regions of stability, and that the most stable regions of the molecule are often, but not necessarily, within the hydrophobic core.

FIG. 9A, FIG. 9B and FIG. 9C illustrate a description of protein structure in terms of thermodynamic environments. FIG. 9A shows the thermodynamic environment classification scheme used herein. Three quantities derived from the output of the COREX algorithm, stability (κ_{i,j}), enthalpy ratio (Hᵣₐₜᵢₒⱼ), and entropy ratio (Sᵣₐₜᵢₒⱼ) describe the thermodynamic environment of each residue. FIG. 9B shows the 12 thermodynamic environments defmed by this classification scheme in a schematic describing protein energetic phase space. Each cube represents a region dominated by certain stability, enthalpy, and entropy characteristics. Every residue position in the protein structures used herein lies somewhere within this phase space. FIG. 9C shows examples of the distribution of thermodynamic environments of (FIG. 9B) in three proteins with varying types and amounts of secondary structure. Note that single secondary structure elements do not exhibit unique thermodynamic environments.

FIG. 10A, FIG. 10B, FIG. 10C, FIG. 10D, FIG. 10E, FIG. 10F, FIG. 10G, FIG. 10H, FIG. 10I, FIG. 10J, FIG. 10K and FIG. 10L show 3D-1D scores relating amino acid types to 12 protein structural thermodynamic environments. The three-letter abbreviation in each panel represents the stability, enthalpic, and entropic descriptor of the thermodynamic environment. Stability is classified into high, medium and low. Entropy and enthalpy are classified into high and low. FIG. 10A represents LHH, which is a protein thermodynamic environment of low stability, high polar/apolar enthalpy ratio, and high conformational entropy/Gibbs' solvation energy ratio. FIG. 10B represents LHL, which is a protein thermodynamic environment of low stability, high polar/apolar enthalpy ratio, and low conformational entropy/Gibbs' solvation energy ratio. FIG. 10C represents LLH, which is a protein thermodynamic environment of low stability, low polar/apolar enthalpy ratio, and high conformational entropy/Gibbs' solvation energy ratio. FIG. 10D represents LLL, which is a protein thermodynamic environment of low stability, low polar/apolar enthalpy ratio, and low conformational entropy/Gibbs' solvation energy ratio. FIG. 10E represents MHH, which is a protein thermodynamic environment of medium stability, high polar/apolar enthalpy ratio, and high conformational entropy/Gibbs' solvation energy ratio. FIG. 10F represents MHL, which is a protein thermodynamic environment of medium stability, high polar/apolar enthalpy ratio, and low conformational entropy/Gibbs' solvation energy ratio. FIG. 10G represents MLH, which is a protein thermodynamic environment of medium stability, low polar/apolar enthalpy ratio, and high conformational entropy/Gibbs' solvation energy ratio. FIG. 10H represents MLL, which is a protein thermodynamic environment of medium stability, low polar/apolar enthalpy ratio, and low conformational entropy/Gibbs' solvation energy ratio. FIG. 10I represents HHH, which is a protein thermodynamic environment of high stability, high polar/apolar enthalpy ratio, and high conformational entropy/Gibbs' solvation energy ratio. FIG. 10J represents HHL, which is a protein thermodynamic environment of high stability, high polar/apolar enthalpy ratio, and low conformational entropy/Gibbs' solvation energy ratio. FIG. 10K represents HLH, which is a protein thermodynamic environment of high stability, low polar/apolar enthalpy ratio, and high conformational entropy/Gibbs' solvation energy ratio. FIG. 10L represents HLL, which is a protein thermodynamic environment of high stability, low polar/apolar enthalpy ratio, and low conformational entropy/Gibbs' solvation energy ratio.

FIG. 11 shows fold-recognition results for 81 protein targets using a scoring matrix composed of thermodynamic information from protein structures. The horizontal axis represents the percentile ranking of the score against the target structure for the sequence corresponding to the target structure. For example, the sequence corresponding to the target cold-shock protein (PDB 1mjc) received the 157^{th} highest score of 3858 sequences against the cold-shock protein thermodynamic profile. This result placed the sequence for the cold-shock protein in the 5th percentile bin in FIG. 11. When aligned with their respective thermodynamic profiles, the majority (44/81) of sequences scored better than 99% of the 3858 sequences in the database.

FIG. 12 shows fold-recognition results for 12 all-beta protein targets using a scoring matrix composed of thermodynamic information from 31 all-alpha protein structures. The horizontal axis represents the percentile ranking of the score against the target structure for the sequence corresponding to the target structure. For example, the sequence corresponding to the all-beta target tendamistat (PDB 1hoe) received the 26^{th} highest score of 3858 sequences against the tendamistat thermodynamic profile. This result placed the tendamistat sequence in the 5^{th} percentile bin in FIG. 5. All 12 sequences corresponding to beta targets scored better against their respective targets than 90% of the 3858 sequences in the database.

### DETAILED DESCRIPTION OF THE INVENTION

It is readily apparent to one skilled in the art that various embodiments and modifications may be made to the invention disclosed in this Application without departing from the scope and spirit of the invention.

As used herein the specification, “a” or “an” may mean one or more. As used herein in the claim(s), when used in conjunction with the word “comprising”, the words “a” or “an” may mean one or more than one. As used herein “another” may mean at least a second or more.

The term “conformation” as used herein refers various nonsuperimposable three-dimensional arrangements of atoms that are interconvertible without breaking covalent bonds.

The term “configuration” as used herein refers to different conformations of a protein molecule that have the same chirality of atoms.

The term “database” as used herein refers to a collection of data arranged for ease of retrieval by a computer. Data is also stored in a manner where it is easily compared to existing data sets.

The term “enthalpy” as used herein refers to a thermodynamic state or environment in which the enthalpy of internal interactions and the hydrophobic entropy change the favor of protein folding, thus enthalpy is a thermodynamic component in the thermodynamic stability of globular proteins. Enthalpy is a ratio of polar and apolar contributions

$$( {H}_{ratio , j} = \frac{Δ {H}_{pol , j}}{Δ {H}_{apol , j}} ) .$$

The term “entropy” as used herein refers to a thermodynamic state or environment in which the conformation entropy change works against folding of proteins. Entropy is a ratio the conformational entropy to total solvation free energy

$$( {S}_{ratio , j} = \frac{Δ {S}_{conf , j}}{Δ {G}_{solv , j}} ) .$$

The term “globular protein” as used herein refers to proteins in which their polypeptide chains are folded into compact structures. The compact structures are unlike the extended filamentous forms of fibrous proteins. A skilled artisan realizes that globular proteins have tertiary structures which comprises the secondary structure elements, e.g., helices, β sheets, or nonregular regions folded in specific arrangements. An example of a globular protein includes, but is not limited to myoglobin.

The term “peptide” as used herein refers to a chain of amino acids with a defined sequence whose physical properties are those expected from the sum of its amino acid residues and there is no fixed three-dimensional structure.

The term “polyamino acids” as used herein refers to random sequences of varying lengths generally resulting from nonspecific polymerization of one or more amino acids.

The term “protein” as used herein refers to a chain of amino acids usually of defined sequence and length and three dimensional structure. The polymerization reaction, which produces a protein, results in the loss of one molecule of water from each amino acid, proteins are often said to be composed of amino acid residues. Natural protein molecules may contain as many as 20 different types of amino acid residues, each of which contains a distinctive side chain.

The term “protein fold” as used herein refers to an organization of a protein to form a structure which constrains individual amino acids to a specific location relative to the other amino acids in the sequence. One of skill in the art realizes that this type of organization of a protein comprises secondary, tertiary and quarternary structures.

The term “thermodynamic environment” as used herein refers to the various thermodynamic components that contribute to the folding process of a protein. For example, stability, entropy and enthalpy thermodynamic environments contribute to the folding of a protein. One skilled in the art realizes that the terms “thermodynamic environment”, “thermodynamic classification” or “thermodynamic component” are interchangeable.

There is a hierarchy of protein structure. The primary structure is the covalent structure, which comprises the particular sequence of amino acid residues in a protein and any posttranslational covalent modifications that may occur. The secondary structure is the local conformation of the polypeptide backbone. The helices, sheets, and turns of a protein's secondary structure pack together to produce the three-dimensional structure of the protein. The three-dimensional structure of many proteins may be characterized as having internal surfaces (directed away from the aqueous environment in which the protein is normally found) and external surfaces (which are in close proximity to the aqueous environment). Through the study of many natural proteins, researchers have discovered that hydrophobic residues (such as tryptophan, phenylalanine, tyrosine, leucine, isoleucine, valine or methionine) are most frequently found on the internal surface of protein molecules. In contrast, hydrophilic residues (such as asparate, asparagine, glutamate, glutamine, lysine, arginine, histidine, serine, threonine, glycine, and proline) are most frequently found on the external protein surface. The amino acids alanine, glycine, serine and threonine are encountered with equal frequency on both the internal and external protein surfaces.

An embodiment of the present invention is a protein database comprising nonhomologous proteins having known residue-specific free energies of folding of the proteins.

One of skill in the art is cognizant that the properties of proteins are governed by their potential energy surfaces. Proteins exist in a dynamic equilibrium between a folded, ordered state and an unfolded, disordered state. This equilibrium in part reflects the interactions between the side chains of amino acid residues, which tend to stabilize the protein's structure, and, on the other hand, those thermodynamic forces which tend to promote the randomization of the molecule.

The present invention utilizes a computational method comprising the step of determining a stability constant from the ratio of the summed probability of all states in the ensemble in which a residue j is in a folded conformation to the summed probability of all states in which j is in an unfolded conformation according the equation,

$${K}_{f , j} = \frac{\sum {P}_{f , j}}{\sum {P}_{nf , j}}$$

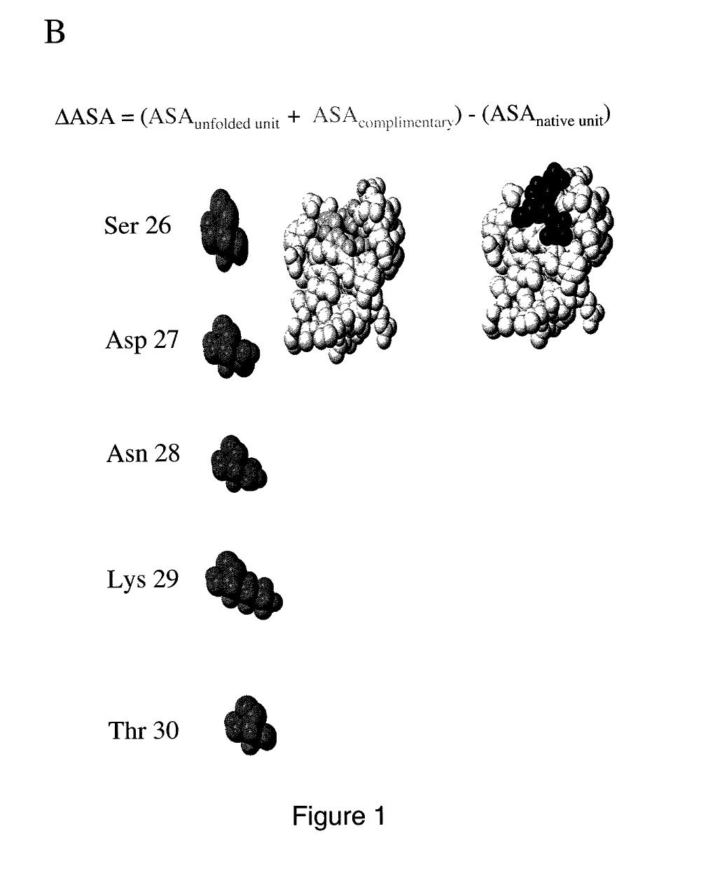

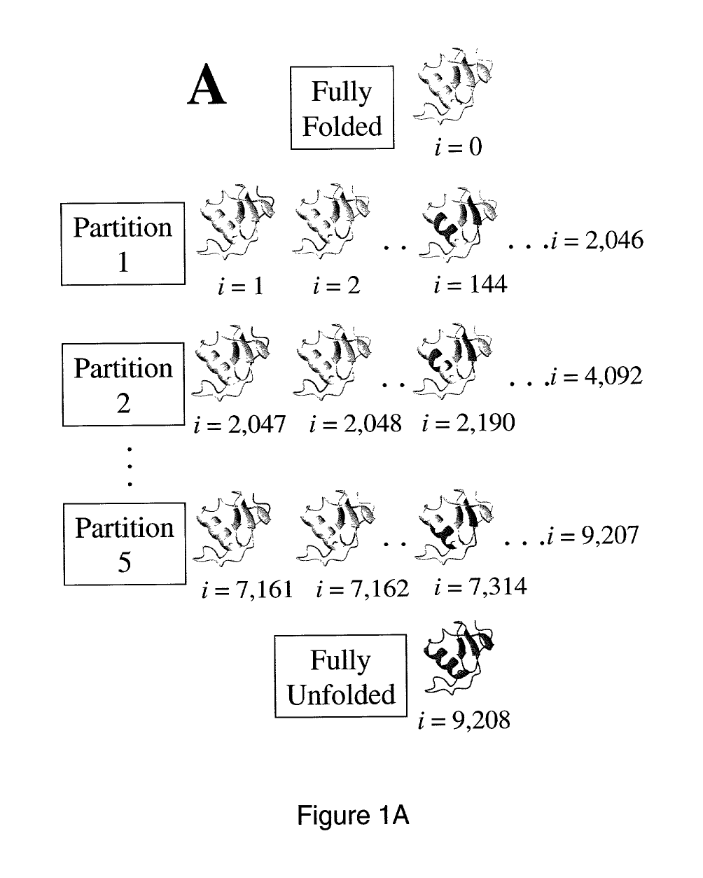

One of skill in the art is cognizant that although the stability constant is defined for each position, the value obtained at each residue is not the energetic contribution of that residue. The stability constant is a property of the ensemble as a whole. For each partially unfolded microstate, the energy difference between it and the fully folded reference state is determined by the energetic contributions of all amino acids comprising the folding units that are unfolded in each microstate, plus the energetic contributions associated with exposing additional (complimentary) surface area on the protein (FIG. 1B). The stability constant thus provides the average thermodynamic environment of each residue, wherein surface area, polarity, and packing are implicitly considered. Thus, the stability constant provides a thermodynamic metric wherein each of these static structural properties is weighted according to its energetic impact at each position.

The stability constants for the residues are arranged into three classifications of stability selected from the group consisting of high, medium and low. Specifically, the residues in the high stability classification comprises phenylalanine, tryptophan and tyrosine. The residues in the low stability classification comprises glycine and proline. The residues in the medium stability classification comprises asparagine and glutamic acid.

In the present invention, the classifications of high, medium and low are determined based upon inspection of the lnκf value for each protein in the selected database. Thus, one of skill in the art is cognizant that these classifications are relative and may vary depending upon the proteins that are selected for the database. One of skill in the art recognizes that these classifications can be subclassified by a variety of other parameters, for example, but not limited to enthalpy and entropy. Thus, any given position in a structure may be represented by two or more parameters, for example, but not limited to low stability (lnκf) and high enthalpy. Yet further, additional parameters can be used to further divide the categories of enthalpy and entropy, for example, but not limited to conformational entropy, solvent entropy, polar enthalpy, apolar enthalpy, polar entropy or apolar entropy Thus, any given position in a structure may have a description such as, but not limited to low stability, high apolar enthalpy, high polar enthalpy, medium conformational entropy and high apolar entropy. One of skill in the art realizes that these classifications allow for better resolution and consequently, better performance in identifying the correct protein fold for a given protein sequence or a portion of a given protein sequence. Further one of skill in the art is cognizant a protein fold refers to the secondary structure of the protein, which includes sheets, helices and turns.

Another specific embodiment of the present invention comprises that the stability constants for the residues are arranged into at least one of the three thermodynamic classification groups selected from the group consisting of stability, enthalpy, and entropy.

Specific embodiments of the present invention provide that the database comprises globular and nonhomologous proteins. A skilled artisan is cognizant that globular proteins are used to study protein folding. It is contemplated that the computational method of the present invention may be used for a variety of globular proteins including but not limiting to glutacorticoid receptor like DNA binding domain, histone, acyl carrier protein like, anti LPS facto/RecA domain, lambda repressor like DNA binding domains, EF hand like, insulin like bacterial Ig/albumin binding, barrel sandwich hybrid, p-loop containing NTP hydrolases, RING finger domain C3HC4, crambin like, ribosomal protein L7/12 C-terminal fragment, cytochrome c, SAM domain like, KH domain, RNA polymerase subunit H, beta-grasp (ubiquitin-like), rubredoxin like, HiPiP, anaphylotoxins (complement system), ferrodoxin like, OB fold, midkine, HMG box, saposin, HPr proteins, knottins, HIV-1 Nef protein fragments, thermostable subdomain from chicken villin, SIS/NS1 RNA binding domain, SH3 like barrel, DNA topoisomerase I domain, IL8 like, de novo designed single chain 3 helix bundle, alpha amylase inhibitor tendamistat, CI2 family of serine protease inhibitors protease inhibitors, protozoan pheromone proteins, ConA like lectins/glucoanases, ovomucoid/PCI-1 like inhibitors, beta clip, snake toxin like and BPTI like. Other globular proteins may be selected from the Protein Data Bank.

One of skill in the art also recognizes that the present invention is not limited to small molecular proteins. A skilled artisan is cognizant that the computational method used in the present invention can be used on larger proteins. Thus, there is not a size limit to the proteins that can be used in the present invention.

Another embodiment of the present invention is a method of developing a protein database comprising the steps of: inputting high resolution structures of proteins; generating an ensemble of incrementally different conformations by combinatorial unfolding of a set of predefined folding units in all possible combinations of each protein; determining the probability of each said conformational state; calculating the residue-specific free energy of each conformational state; and classifying a stability constant into at least one thermodynamic environment selected from the group consisting of stability, enthalpy, and entropy.

In specific embodiments, the generating step comprises dividing the proteins into folding units by placing a block of windows over the entire sequence of the protein and sliding the block of windows one residue at a time.

One of skill in the art is cognizant that the division of a protein into a given number of folding units is a partition. Thus, to maximize the number of partially folded states, different partitions are used in the analysis. The partitions can be defined by placing a block of windows over the entire sequence of the protein. The folding units are defmed by the location of the windows irrespective of whether they coincide with specific secondary structure elements. By sliding the entire block of windows one residue at a time, different partitions of the protein are obtained. For two consecutive partitions, the first and last amino acids of each folding unit are shifted by one residue. This procedure is repeated until the entire set of partitions has been exhausted. In specific embodiments, windows of 5 or 8 amino acid residues are used. One of skill in the art realizes that approximately 10⁵ partially folded conformations can be generated using the COREX algorithm. This value can be altered by increasing or decreasing the window size and the size of the protein. For example, for the proteins λ6-85, chymotrypsin inhibitor 2 and barnase, windows sizes of 5, 5, 8 and amino acid residues results in 2.6×10⁵, 0.4×10⁵, and 1.1×10⁵ partially folded conformations, respectively.

In further embodiments, the determining step comprises determining the free energy of each of the conformational states in the ensemble; determining the Boltzmann weight [Kᵢ=exp(−ΔGᵢ/RT)] of each state; and determining the probability of each state using the equation,

$${P}_{i} = \frac{{K}_{i}}{\sum {K}_{i}^{′}}$$

Yet further, the calculating step comprises determining the energy difference between all microscopic states in which a particular residue is folded and all such states in which it is unfolded using the equation,

ΔG_{f,j}=−RT·lnκ_{f,j}

One of skill in the art is aware that the COREX algorithm generates a large number of partially folded states of a protein from the high resolution crystallographic or NMR structure (Hilser & Freire, 1996; Hilser & Freire, 1997 and Hilser et al., 1997). In this algorithm, the high resolution structure is used as a template to approximate the ensemble of partially folded states of a protein. Thus, the protein is considered to be composed of different folding units. The partially folded states are generated by folding and unfolding these units in all possible combinations. There are two basic assumptions in the COREX algorithm: (1) the folded regions in partially folded states are native-like; and (2) the unfolded regions are assumed to be devoid of structure or lacking structure. Thermodynamic quantities, e.g., ΔH, ΔS, ΔCp, and ΔG, partition function and probability of each state (Pᵢ) are evaluated using an empirical parameterization of the energetics (Murphy & Freire, 1992; Gomez et al., 1995; Hilser et al., 1996; Lee et al., 1994; D'Aquino et al., 1996; and Luque et al., 1996).

Yet further, a skilled artisan is cognizant that the residue specific equilibrium provide quantitative agreement with those obtained experimentally from amide hydrogen exchange experiments, e.g., hydrogen protection factors (Hilser & Freire, 1996; Hilser & Freire, 1997; and Hilser et al., 1997).

One of skill in the art realizes that while the residue stability constants are purely thermodynamic quantities defined for all residues, the protection factors also contain non-thermodynamic contributions and are defined for a subset of residues.

Another embodiment of the present invention is a method of identifying a protein fold comprising determining the distribution of amino acid residues in different thermodynamic environments corresponding to a known protein structure. More particularly, determining the distribution of amino acid residues comprises constructing scoring matrices derived of thermodynamic information. Specifically, the scoring matrices are derived from COREX thermodynamic information, such as stability, enthalpy, and entropy. Thus, COREX-derived thermodynamic descriptors can be used to identify sequences that correspond to a specific fold.

A skilled artisan recognizes that the COREX algorithm provides a means of estimating the energetic variability in the native state of proteins, and uses this information to illuminate the relation between amino acid sequence and protein structure. Therefore, the thermodynamic information obtained by the COREX algorithm represents a fundamental descriptor of proteins that transcends secondary structure classifications.

Protein folds can be considered as one of the most basic molecular parts. A skilled artisan recognizes that the properties related to protein folds can be divided into two parts, intrinsic and extrinsic. The intrinsic properties relates to an individual fold, e.g., its sequence, three-dimensional structure and function. Extrinsic properties relates to a fold in the context of all other folds, e.g., its occurrence in many genomes and expression level in relation to that for other folds.

Further, one of skill in the art realizes that other methods well known in the art can be used to develop protein databases for example, but not limited to Monte Carlo sampling method. The Monte Carlo sampling method is well known and used in the art (Pan et al., 2000).

### EXAMPLES

The following examples are included to demonstrate preferred embodiments of the invention. It should be appreciated by those skilled in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the concept, spirit and scope of the invention.

### Example 1

### Selection of Proteins Used in Dataset

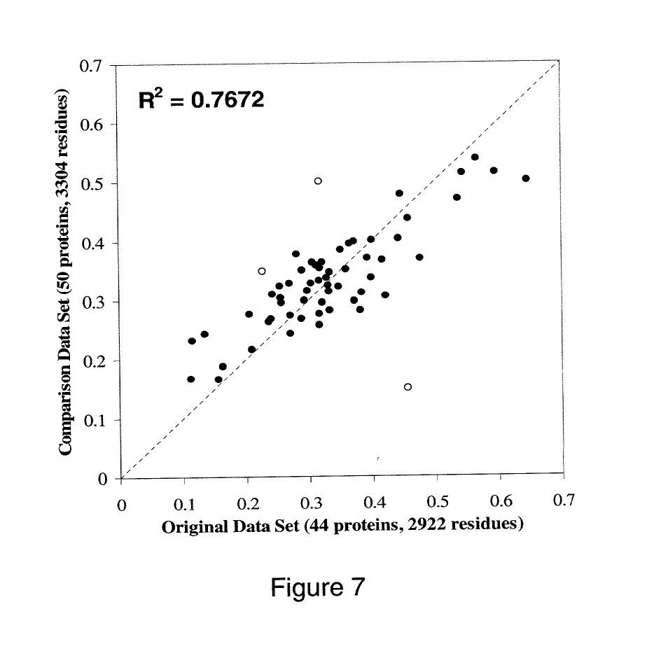

A database of 44 proteins, 2922 residues total (Table 1), was selected from the Protein Data Bank on the basis of biological and computational criteria. The two biological criteria were that the proteins be globular and nonhomologous with every other member of the set as ascertained by SCOP (Murzin et al., 1995). The first computational criterion was that the proteins be small (less than about 90 residues), because the CPU time and data storage needs of an exhaustive COREX calculation increased exponentially with the chain length. The second computational criterion was that the structures be mostly devoid of ligands, metals, or cofactors, as the COREX energy function was not parameterized to account for the energetic contributions of non-protein atoms. The database was comprised of 24 x-ray structures, whose resolution ranged from 2.60 to 1.00 Å (median value of 1.65 Å). Twenty NMR structures completed the database. An independent database of 50 proteins (3304 residues total) that were not included in the above set, was created from the PDBSelect database (Hobohm & Sander, 1996). This second database was used as a control to check the results obtained from the first database, as shown in FIG. 7.

TABLE 1
SCOP Classifications and Sequence Data for 44 Proteins Used in the Database

| | PDB | PDB | | SEQUENCE | NUMBER OF |
|---|---|---|---|---|---|
| NUMBER | ID | LENGTH^{a} | SCOP FOLD^{b} | LENGTH^{c} | SEQUENCES^{d} |
| 1 | 1a7I | 60 | Glutacorticoid receptor like DNA binding domain | 81 | 20 |
| 2 | 1a7w | 68 | Histone | 69 | 17 |
| 3 | 1a8o | 70 | Acyl carrier protein like | 70 | 22 |
| 4 | 1aa3 | 63 | Anti LPS factor/RecA domain | 63 | 12 |
| 5 | 1adr | 76 | Lambda repressor like DNA binding domains | 76 | 35 |
| 6 | 1ak8 | 76 | EF hand like | 76 | 35 |
| 7 | 1b9g | 57 | Insulin like | 57 | 12 |
| 8 | 1bdd | 60 | Bacterial Ig/albumin binding | 60 | 32 |
| 9 | 1bdo | 80 | Barrel sandwich hybrid | 80 | 14 |
| 10 | 1cly | 77 | P-loop containing NTP hydrolases | 77 | 25 |
| 11 | 1chc | 68 | RING finger domain C3HC4 | 68 | 21 |
| 12 | 1cnr | 46 | Crambin like | 46 | 30 |
| 13 | 1ctf | 68 | Ribosomal protein L7/12 C-terminal fragment | 74 | 14 |
| 14 | 1ctj | 89 | Cytochrome c | 89 | 17 |
| 15 | 1doq | 69 | SAM domain like | 69 | 17 |
| 16 | 1dt4 | 73 | KH domain | 73 | 16 |
| 17 | 1hmj | 68 | RNA polymerase subunit H | 78 | 8 |
| 18 | 1igd | 61 | Beta-grasp (ubiquitin-like) | 61 | 12 |
| 19 | 1iro | 53 | Rubredoxin like | 54 | 16 |
| 20 | 1isu | 62 | HiPiP | 62 | 30 |
| 21 | 1kjs | 74 | Anaphylotoxins (complement system) | 74 | 14 |
| 22 | 1kp6 | 79 | Ferrodoxin like | 79 | 24 |
| 23 | 1mjc | 69 | OB fold | 69 | 17 |
| 24 | 1mkn | 59 | Midkine | 59 | 9 |
| 25 | 1nhm | 79 | HMG box | 81 | 20 |
| 26 | 1nkl | 78 | Saposin | 78 | 8 |
| 27 | 1ptf | 87 | HPr proteins | 87 | 33 |
| 28 | 1ptx | 64 | Knottins | 64 | 19 |
| 29 | 1qa4 | 56 | HIV-1 Nef protein fragments | 57 | 12 |
| 30 | 1qqv | 67 | Thermostable subdomain from chicken villin | 67 | 20 |
| 31 | 1r1b | 56 | SIS/NS1 PNA binding domain | 59 | 9 |
| 32 | 1sem | 58 | SH3 like barrel | 58 | 23 |
| 33 | 1vcc | 77 | DNA topoisomerase I domain | 77 | 25 |
| 34 | 1vmp | 71 | IL8 like | 71 | 25 |
| 35 | 2a3d | 73 | De novo designed single chain 3 helix bundle | 73 | 16 |
| 36 | 2ait | 74 | Alpha amylase inhibitor tendamistat | 74 | 14 |
| 37 | 2ci2 | 65 | CI2 family of serine protease inhibitors | 83 | 8 |
| 38 | 2er1 | 40 | Protozoan pheromone proteins | 40 | 24 |
| 39 | 2ezh | 65 | DNA/RNA binding 3-helical bundle | 75 | 17 |
| 40 | 21a1 | 47 | ConA like lectins/glucanases | 52 | 6 |
| 41 | 2ovo | 56 | Ovomucoid/PCI-1 like inhibitors | 56 | 25 |
| 42 | 2spg | 66 | Beta clip | 66 | 31 |
| 43 | 3ebx | 62 | Snake toxin like | 62 | 30 |
| 44 | 6pti | 56 | BPTI like | 58 | 23 |

^{a}The number of residues for which coordinates are reported in the PDB entry.
^{b}The structural classification for determining extent of homology as found in the SCOP database (Murzin et al., 1995).
^{c}The number of residues in the entire amino acid sequence as given in the PDB entry. All amino acid sequences contained in the fold-recognition library of a given target structure were of this length.
^{d}The number of amino acid sequences contained in the fold-recognition library of a given target structure and represents the total number of monomeric sequences in the PDB with lengths identical to the value in the “Sequence Length” column.

### Example 2

### Computational Details

The database of 44 nonhomologous proteins (Table 1) was analyzed using the COREX algorithm. The COREX algorithm (Hilser & Freire, 1996) was run with a window size of five residues on each protein in the database. The minimum window size was set to four, and the simulated temperature was 25° C.

Briefly, COREX generated an ensemble of partially unfolded microstates using the high-resolution structure of each protein as a template (Hilser & Freire, 1996). This was facilitated by combinatorially unfolding a predefined set of folding units (i.e., residues 1-5 are in the first folding unit, residues 6-10 are in the second folding unit, etc.). By means of an incremental shift in the boundaries of the folding units, an exhaustive enumeration of the partially unfolded species was achieved for a given folding unit size. The entire procedure is shown schematically in FIG. 1A for ovomucoid third domain (OM3), one of the proteins in the database (PDB accession code 2ovo).

For each microstate i in the ensemble, the Gibbs free energy was calculated from the surface area-based parameterization described previously (D'Aquino, 1996; Gomez, 1995; Xie, 1994; Baldwin, 1986; Lee, 1994; Habermann, 1996). The Boltzmann weight of each microstate [i.e., Ki=exp(−ΔGᵢ/RT)] was used to calculate its probability:

$${P}_{i} = \frac{{K}_{i}}{\sum {K}_{i}} \qquad ( 1 )$$

where the summation in the denominator is over all microstates. From the probabilities calculated in Equation 1, an important statistical descriptor of the equilibrium was evaluated for each residue in the protein. Defined as the residue stability constant, κ_{fj}, this quantity was the ratio of the summed probability of all states in the ensemble in which a particular residue j was in a folded conformation (ΣP_{f,j}) to the summed probability of all states in which j was in an unfolded conformation (ΣP_{nf,j}):

$${κ}_{f , j} = \frac{\sum {P}_{f , j}}{\sum {P}_{nf , j}} \qquad ( 2 )$$

From the stability constant, a residue-specific free energy was written as:

ΔG_{f,j}=−RT·lnκ_{f,j} (3)

Equation 3 reflects the energy difference between all microscopic states in which a particular residue was folded and all such states in which it is unfolded.

The Gibbs energy for each microstate i relative to the fully folded structure was calculated using Equation 4:

ΔGi=ΔHi, solvation−T(ΔSi, salvation+WΔSi, conformational) (4)

where the calorimetric enthalpy and entropy of solvation were parameterized from polar and apolar surface exposure, and the conformational entropy was determined as described previously (Hilser & Freire, 1996). The maximum stability for each protein was normalized to a common arbitrary value of approximately 6.2 kcal/mol (max lnκ_{f}=10.4) by adjusting its conformational entropy factor, W, in Equation 4. The average entropy factor required for the normalization was 0.81±0.19 (mean±s.d.) over the 44 proteins. It was an empirical observation that adjustment of a stable protein's conformational entropy factor did not change the relative patterns of high and low stability regions in the structure.

### Example 3

### Comparison of Residue Stability Constant to Hydrogen Exchange Protection Factors

Prediction of the hydrogen exchange protection factors of the residues that exchange protons was performed by calculation of the ensemble of P_{f,j} and P_{f,cx,j} values.

Briefly, the protection factor for any given residue j was defined as the ratio of the sum of the probabilities of the states in which residue j was closed, to the sum of the probabilities of the states in which residue j was open:

$${PF}_{j} = \frac{\sum {P}_{i}}{\sum {P}_{i}} = \frac{{P}_{closed} , j}{{P}_{open} , j} \qquad ( 5 )$$

The statistical definition of the protection factors has the same form as that of the stability constants (equation (2)) and was expressed in terms of the folding probabilities as follows:

$${PF}_{j} = \frac{{P}_{f , j} - {P}_{f , xc , j}}{{P}_{n , f , j} + {P}_{f , xc , j}} \qquad ( 6 )$$

The correction term P_{f,xc,j} was the sum of the probabilities of all states in which residue j was folded, yet exchange competent.

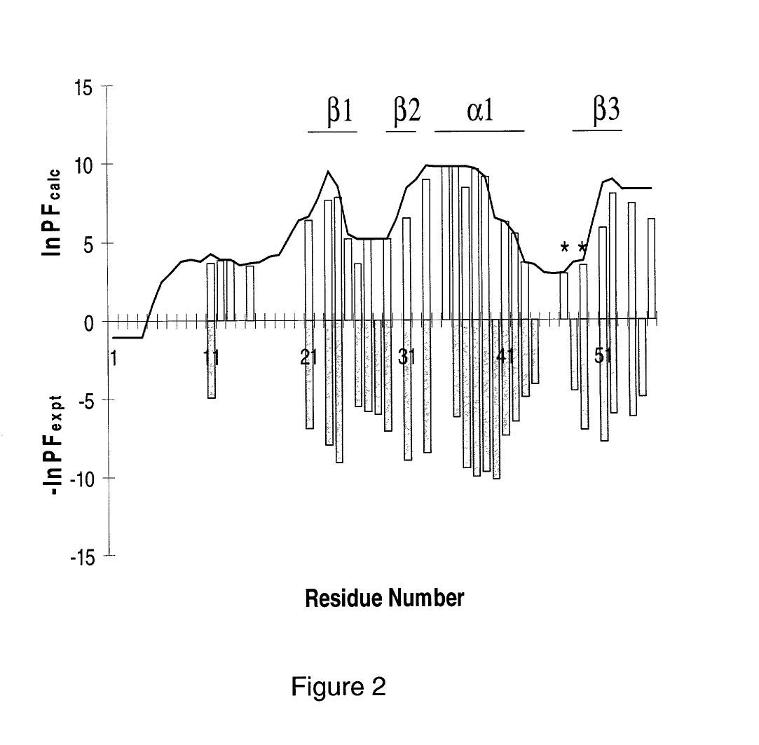

FIG. 2 shows the comparison of hydrogen exchange protection factors predicted from COREX data with experimental values for OM3. The agreement in the location and relative magnitude of the protection factors with the stability constants for this and other proteins suggested that the calculated native state ensemble provided a good description of the actual ensemble (Hilser & Freire, 1996). It naturally follows that the residue stability constants of a particular protein provided a good description of the thermodynamic environment of each residue in that structure.

Further inspection of FIG. 2 revealed another important feature in the pattern of residue stability constants. Namely, the stability constants varied significantly across a given secondary structural element, as observed for alpha helix 1 of OM3. The protection factors (and stability constants) were high at the N-terminal region of helix 1, but decreased over the length of the helix. This indicated that secondary structure, or other structural classifications, do not obligatorily coincide with thermodynamic classifications. This result has potentially important consequences for cataloging propensities of amino acids in different environments. For example, in OM3 two threonine residues were located in different structural environments; Thr 47 was part of the loop that follows alpha helix 1, while Thr 49 was part of beta strand 3. In spite of the different structural environments for the two threonine residues, the stability constants and, more importantly, the experimental protection factors demonstrated that both residues, to a first approximation, share the same thermodynamic environment.

### Example 4

### Binning of Residue Stability Constants

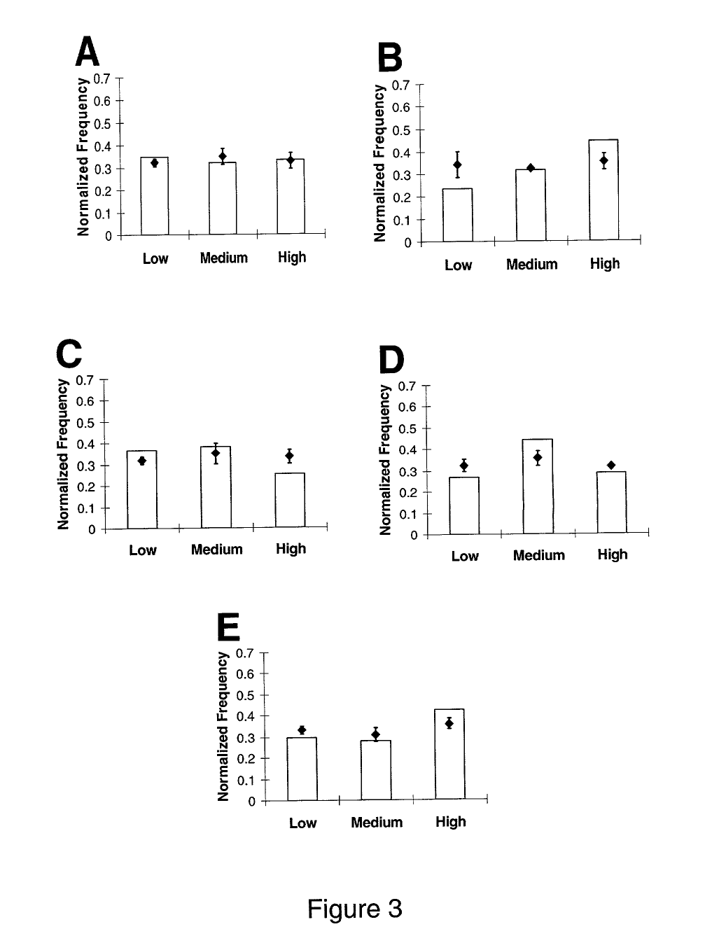

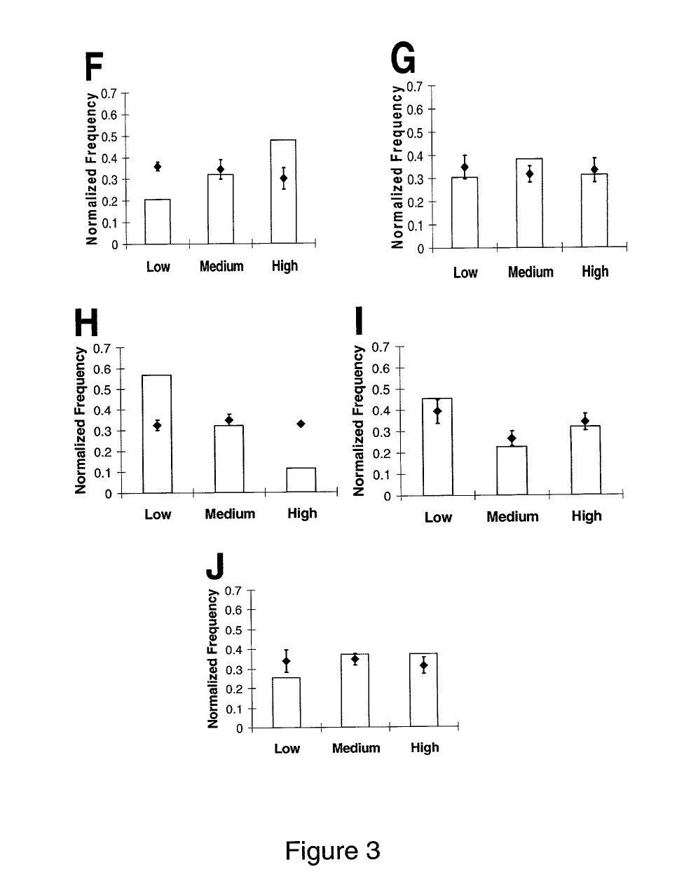

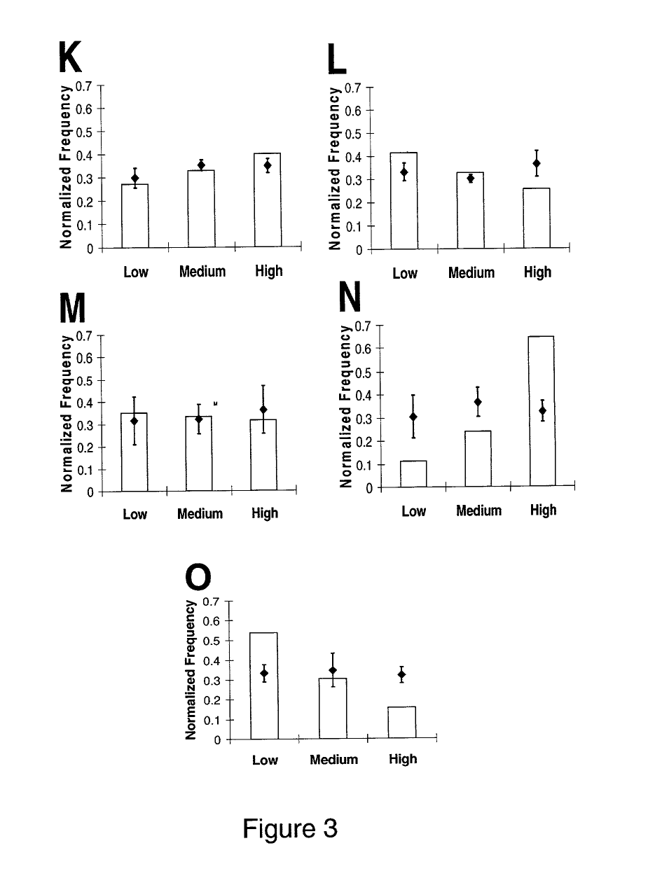

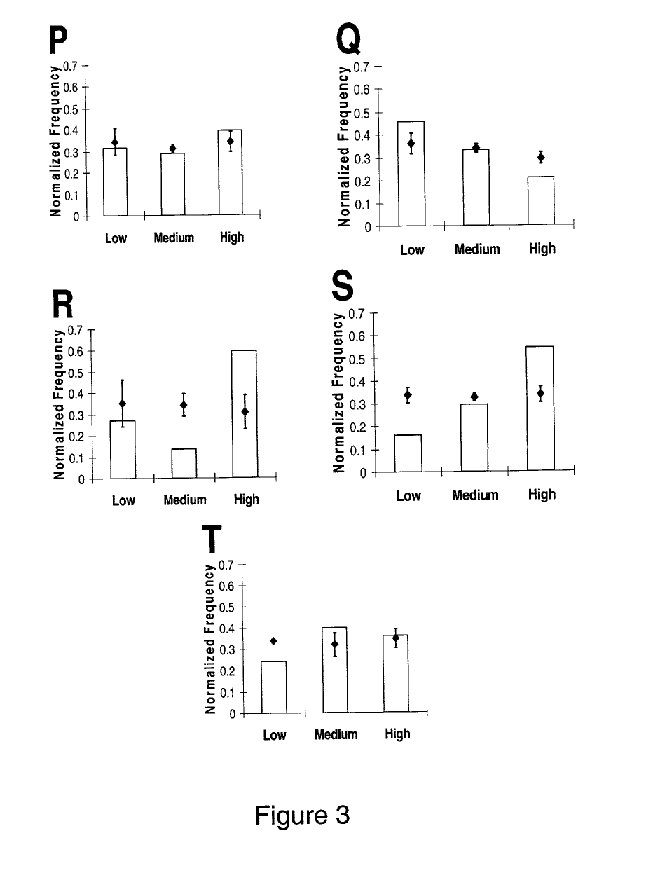

Inspection of each protein's inκ_{f} data indicated that these were the three stability classes: high, medium, and low stability. The cutoffs for each stability class were adjusted so that an approximately equal number of residues in the database fell in each class (Table 2). The low stability category was defined as lnκ_{f}<=3.99, the medium stability category was defined as 3.99<lnκ_{f}<=7.14, and the high stability category was defined as lnκ_{f}>7.14. Statistics of amino acid type as a function of each of these stability categories were tabulated (Table 2), and normalized histograms of these numbers are shown in FIG. 3A-FIG. 3T.

TABLE 2
Statistics of lnκ_{f} Values for 2922 Residues in the Database^{a}

| | Low | Medium | High | |
|---|---|---|---|---|
| Residue | (lnκ_{f} <= | (3.99 < lnκ_{f} <= | (7.14 < | Row |
| Type | 3.99) | 7.14) | lnκ_{f} ) | Total |
| Ala | 95 | 88 | 91 | 274 |
| Arg | 33 | 43 | 63 | 139 |
| Asn | 46 | 47 | 33 | 126 |
| Asp | 42 | 69 | 45 | 156 |
| Cys | 36 | 34 | 51 | 121 |
| Gln | 22 | 34 | 51 | 107 |
| Glu | 68 | 86 | 70 | 224 |
| Gly | 125 | 71 | 25 | 221 |
| His | 20 | 10 | 14 | 44 |
| Ile | 36 | 55 | 54 | 145 |
| Leu | 58 | 70 | 87 | 215 |
| Lys | 99 | 78 | 61 | 238 |
| Met | 20 | 19 | 18 | 57 |
| Phe | 11 | 23 | 62 | 96 |
| Pro | 71 | 41 | 22 | 134 |
| Ser | 46 | 41 | 58 | 145 |
| Thr | 70 | 51 | 32 | 153 |
| Trp | 10 | 5 | 22 | 37 |
| Tyr | 15 | 27 | 50 | 92 |
| Val | 48 | 79 | 71 | 198 |
| Column Total | 971 | 971 | 980 | 2922 |

^{a}The values in this table were used to compute the normalized histograms shown in FIG. 3A-FIG. 3T. In addition, these values (minus the values for a given target protein) were used to compute the lnκ_{f} scoring matrices.

Striking asymmetries were often observed for the histograms of certain amino acids across the three stability environments, and these asymmetries were well outside the standard deviation of the average of three random data sets. For example, the aromatic amino acids Phe, Trp, and Tyr were mostly found in high stability environments, while Gly and Pro were overwhelmingly found in low stability environments. In contrast, other residues such as Ala, Met, and Ser exhibited distributions that did not significantly differ from randomized data.

Although the acidic residues Asp and Glu shared a slight tendency to be found in medium stability environments, it was observed that several amino acid pairs having nominally similar chemical characteristics partition differently in the stability environments. For example, the basic residues Arg and Lys exhibited opposite stability characteristics: the counts for Arg increased as the stability class increased, but the counts for Lys decreased as a function of stability class. While Asn was found less often in high stability environments, Gln was found more often in them. Although the distribution for Ser did not differ significantly from the randomized data, Thr occurred more often in low stability environments and less often in high stability environments. Somewhat surprisingly, the aliphatic amino acids Ile, Leu, and Val did not show a general pattern, except perhaps a slight disfavoring of low stability environments.

### Example 5

### Calculation of Average Native State Side Chain Area Surface Exposure

Average side chain area surface area exposure of residue j over a window size of five residues, ASA_{averagej}, was calculated using Equation 7:

$${ASA}_{average , j} = \frac{{\sum }_{i = j - 2}^{i = j + 2} {ASA}_{native , j}}{5} \qquad ( 7 )$$

Because Equation 7 was undefined for the first and last two residues in each protein, these four residues were ignored in the binning. The cutoffs for each side chain area class were adjusted so that an approximately equal number of residues fell in each class. The low exposure category was defined as ASA_{average,j}<=43.31 Å², the medium exposure category was defined as 43.31 Å²<ASA_{average,j}<=59.86 Å², and the high exposure category was defined as ASA_{average,j}>59.86 Å².

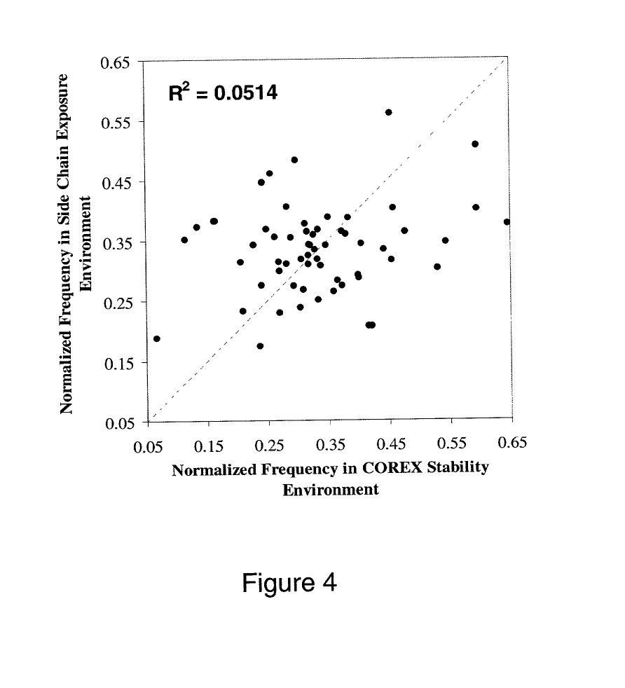

As shown in FIG. 4, frequencies of amino acids found in COREX stability environments were not correlated to frequencies of amino acids in exposed surface area environments. This was important as it suggested that the thermodynamic information calculated by the COREX algorithm was not simply monitoring a static property of the structure, but instead was capturing a property of the native state ensemble as a whole.

### Example 6

### Random DataSets

For comparison to the COREX and DSSP data sets from the 44 non-homologous proteins in the database, control data sets were constructed by randomizing (i.e., shuffling) the calculated stability and the secondary structure data. The random data sets therefore contained the same amino acid composition, counts of high, medium, and low stabilities, and types of secondary structure, as the real data sets. However, any correlation between residue type or secondary structural class was presumably destroyed by randomization. To assess internal variability of the data due to differing numbers of counts of each residue type, the results from three randomized data sets were averaged and standard deviations calculated; these data are plotted in FIG. 3A-FIG. 3T.

### Example 7

### Construction of Scoring Matrices

The scoring matrices were calculated as log-odds probabilities of finding residue type j in structural environment k, as described below and in (Bowie et al., 1991). The matrix score, S_{j,k}, was defined as:

$${S}_{j , k} = ln \frac{{P}_{j} \vert k}{{P}_{k}} \qquad ( 8 )$$

In Equation 8, Pⱼ|k was the probability of finding a residue of type j in stability class k (i.e., number of counts of residue type j in stability class k divided by the total number of counts of residue type j), and Pₖ was the probability of finding any residue in the database in stability environment k (i.e., number of residues in stability class k, regardless of amino acid type, divided by the total number of residues in the entire database, regardless of amino acid type). The structural environment was described by either COREX stability information (high, medium, or low lnκ_{f}), or DSSP secondary structure (alpha, beta, or other) as given in the target's PDB entry. The fold recognition target was removed from the database, and the remaining 43 proteins were used to calculate the scores; therefore, information about the target was never included in the scoring matrix. The values in Tables 3A and 3B are the average ± standard deviation of all 44 individual scoring matrices.

TABLE 3
Average
3D-1D Scoring Matrices Derived from Inκ_{f} and Secondary Structure Information^{a}

| A. | W | F | Y | L | I | V | M | A | G | P |
|---|---|---|---|---|---|---|---|---|---|---|
| Low^{b} | −0.21 | −1.06 | −0.71 | −0.21 | −0.29 | −0.32 | 0.05 | 0.04 | 0.53 | 0.47 |
| | ±0.05 | ±0.04 | ±0.04 | ±0.02 | ±0.02 | ±0.02 | ±0.03 | ±0.02 | ±0.01 | ±0.01 |
| Medium | −0.90 | −0.33 | −0.12 | −0.02 | 0.13 | 0.18 | 0.00 | −0.03 | −0.03 | −0.08 |
| | ±0.06 | ±0.02 | ±0.03 | ±0.01 | ±0.01 | ±0.01 | ±0.03 | ±0.01 | ±0.02 | ±0.02 |
| High | 0.57 | 0.66 | 0.48 | 0.19 | 0.10 | 0.07 | 0.06 | −0.01 | −1.09 | −0.71 |
| | ±0.02 | ±0.01 | ±0.02 | ±0.01 | ±0.01 | ±0.01 | ±0.02 | ±0.01 | ±0.03 | ±0.03 |
| | C | T | S | Q | N | E | D | H | K | R |
| Low | −0.11 | 0.32 | −0.05 | −0.48 | 0.09 | −0.09 | −0.21 | 0.31 | 0.22 | −0.34 |
| | ±0.02 | ±0.01 | ±0.02 | ±0.03 | ±0.02 | ±0.01 | ±0.02 | ±0.03 | ±0.02 | ±0.03 |
| Medium | −0.17 | 0.00 | −0.16 | −0.04 | 0.12 | 0.14 | 0.29 | −0.38 | −0.01 | −0.07 |
| | ±0.03 | ±0.02 | ±0.02 | ±0.02 | ±0.02 | ±0.01 | ±0.01 | ±0.04 | ±0.02 | ±0.02 |
| High | 0.23 | −0.47 | 0.18 | 0.35 | −0.25 | −0.07 | −0.15 | −0.05 | −0.27 | 0.30 |
| | ±0.02 | ±0.03 | ±0.01 | ±0.02 | ±0.03 | ±0.01 | ±0.02 | ±0.04 | ±0.02 | ±0.02 |
| B. | W | F | Y | L | I | V | M | A | G | P |
| Alpha^{c} | −0.20 | 0.04 | −0.04 | 0.30 | 0.11 | −0.22 | 0.16 | 0.42 | −0.85 | −1.08 |
| | ±0.04 | ±0.03 | ±0.03 | ±0.01 | ±0.02 | ±0.02 | ±0.03 | ±0.01 | ±0.03 | ±0.03 |
| Beta | 0.57 | 0.78 | 0.66 | −0.22 | 0.52 | 0.67 | −0.31 | −0.65 | −0.44 | −0.47 |
| | ±0.04 | ±0.02 | ±0.03 | ±0.03 | ±0.02 | ±0.02 | ±0.06 | ±0.03 | ±0.03 | ±0.04 |
| Other | −0.15 | −0.55 | −0.34 | −0.17 | −0.36 | −0.21 | −0.02 | −0.20 | 0.40 | 0.44 |
| | ±0.03 | ±0.03 | ±0.02 | ±0.01 | ±0.03 | ±0.02 | ±0.02 | ±0.02 | ±0.01 | ±0.01 |
| | C | T | S | Q | N | E | D | H | K | R |
| Alpha | −0.05 | −0.52 | −0.34 | 0.35 | −0.63 | 0.35 | −0.00 | −0.37 | 0.13 | 0.19 |
| | ±0.03 | ±0.03 | ±0.02 | ±0.02 | ±0.03 | ±0.01 | ±0.02 | ±0.05 | ±0.01 | ±0.02 |
| Beta | 0.08 | 0.54 | −0.20 | 0.20 | −0.49 | −0.49 | −0.63 | 0.20 | −0.55 | −0.11 |
| | ±0.04 | ±0.02 | ±0.03 | ±0.02 | ±0.03 | ±0.03 | ±0.05 | ±0.05 | ±0.03 | ±0.03 |
| Other | 0.00 | 0.03 | 0.22 | −0.43 | 0.36 | −0.15 | 0.15 | 0.12 | 0.05 | −0.11 |
| | ±0.02 | ±0.01 | ±0.01 | ±0.02 | ±0.01 | ±0.01 | ±0.01 | ±0.02 | ±0.01 | ±0.02 |

^{a}Each of the 44 targets used in the fold-recognition experiments had an individual 3D-1D scoring matrix that did not include information about the target. Consequently, matrix scores are reported as the average (large bold numbers) ± standard deviation (small numbers) of 44 values.
^{b}The boundaries for the stability categories were defined as follows: low stability was 1nκ_{f} <= 3.99, medium stability was 3.99 < 1n□_{f} <= 7.14, high stability was 7.14 < 1nκ_{f}, as described in the text.
^{c}DSSP secondary structure (Kabsch & Sander, 1983) was used as given in each protein's PDB entry.

The scoring matrices derived from COREX stability and secondary structure, averaged over all 44 target proteins, are shown in Tables 3A and 3B, respectively. The stability matrix scores faithfully reflected the histograms shown in FIG. 3A-FIG. 3T; for example, Gly and Pro scored unfavorably in high stability environments but scored favorably in low stability environments. Similarly, the secondary structure matrix scores followed intuitive notions of secondary structure propensity; for example, Ala scored positively in helical environments, the aromatics scored positively in beta environments, and Gly and Pro scored negatively in both alpha and beta environments. The standard deviations in both matrices were generally small as compared to the magnitude of the scores, suggesting that the scores were not affected by the removal of any one protein from the database.

### Example 8

### Fold-Recognition Details

Fold-recognition experiments were based on the profile method pioneered by Eisenberg and co-workers (Gribskov et al., 1987; Bowie et al., 1991).

Briefly, the method characterized each residue position of a target protein in terms of a structural environment score derived from analysis of a database of known structures. The resulting profile of the target protein was then optimally aligned to each member of a library of amino acid sequences by maximizing the score between the sequence and the profile. Two structural environment scoring schemes were developed: one based on calculated COREX stability, and one based on DSSP secondary structure (Kabsch & Sander, 1983) as contained in each target protein's PDB file. Each scoring scheme had three dimensions as a function of the 20 amino acids: high, medium, and low stability for COREX scoring, or alpha, beta, and other for secondary structure scoring. Two alignment algorithms were used: a local scheme (Smith & Waterman, 1981) as implemented in the PROFILESEARCH software package (Bowie et al., 1991), and a global scheme. The global alignment scheme simply paired the first residue of an amino acid sequence with the first position of a target profile, with no allowance for gaps. This scheme was possible because the amino acid sequence lists against which the targets were threaded only included sequences of identical length to each target corresponding to monomeric structures from the PDB. The total number of identical length sequences for each target ranged from 6 to 35, with an average of 19±8 sequences per target (Table 1). No attempt was made to optimize the gap opening and extension penalties for the local algorithm; in all cases these were the defaults given in the PROFILESEARCH package, 0.1 and 0.05, respectively.

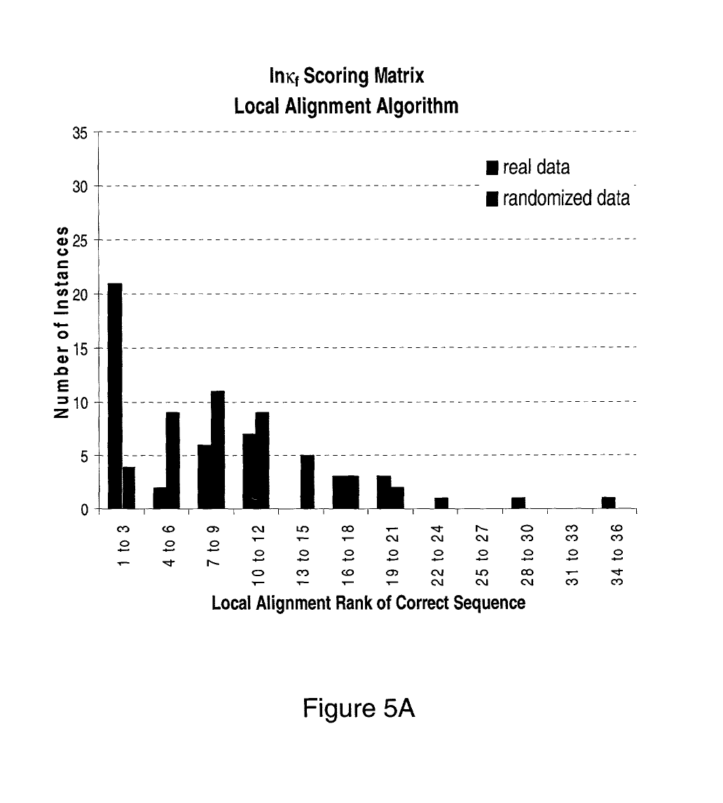

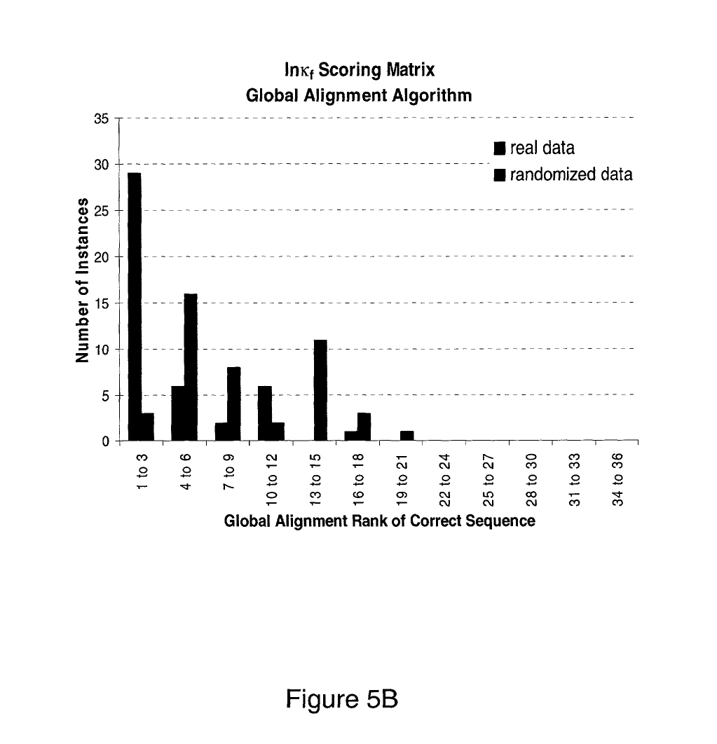

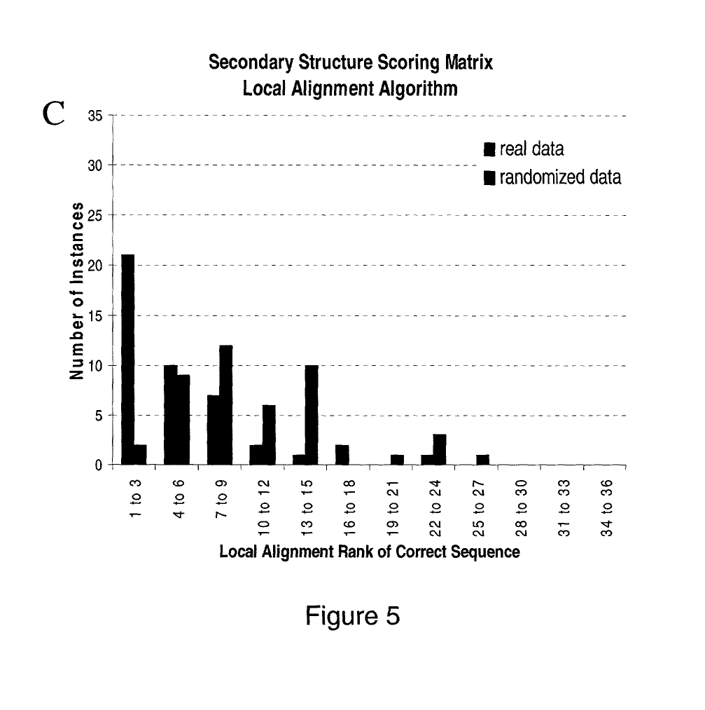

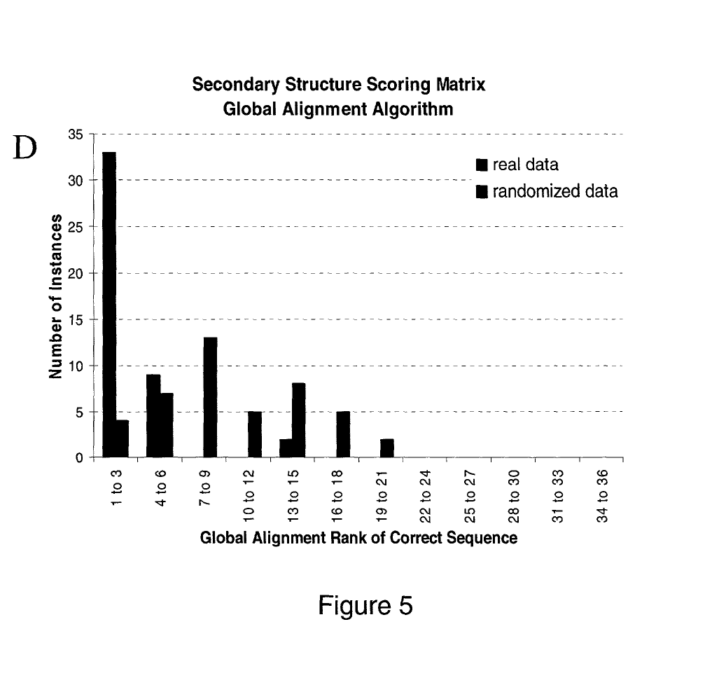

The results of the fold recognition experiments are shown in FIG. 5A, FIG. 5B, FIG. 5C and FIG. 5D, and at least three conclusions are drawn from this data. First, scoring matrices composed of either COREX stability or DSSP secondary structure data performed better than randomized data sets in matching a structural target to its amino acid sequence. In FIG. 5A, FIG. 5B, FIG. 5C and FIG. 5D, the results for COREX data are stacked toward the left (successful) side of the rankings, while the randomized data approaches a bell-shaped distribution with a maximum near the median of the size of the sequence datasets (approximately 10 for the mean size of 19 sequences). Second, for both COREX and DSSP scoring matrices, the global algorithm (which took the entire amino acid sequence into account) performed significantly better than the local algorithm (which generally aligned only a subset of the sequence). Third, the total number of targets falling in the most successful bin was similar for both the COREX stability and secondary structure matrices, suggesting that COREX stability propensities alone contained a comparable amount of information to secondary structure propensities.

Because the local alignment algorithms used here compute a score without returning the complete alignment of profile to sequence, high scores may have been possible from non-structurally significant local alignments. In other words, it is possible that a correct sequence may have scored well against its corresponding target structure without having placed the individual amino acids in their correct positions within the structure. The use of the global alignment in conjunction with amino acid sequences of identical length partially alleviated this problem, as no misalignment was allowed in the global scheme.

### Example 9

### Successful Alignment Based on COREX Stability

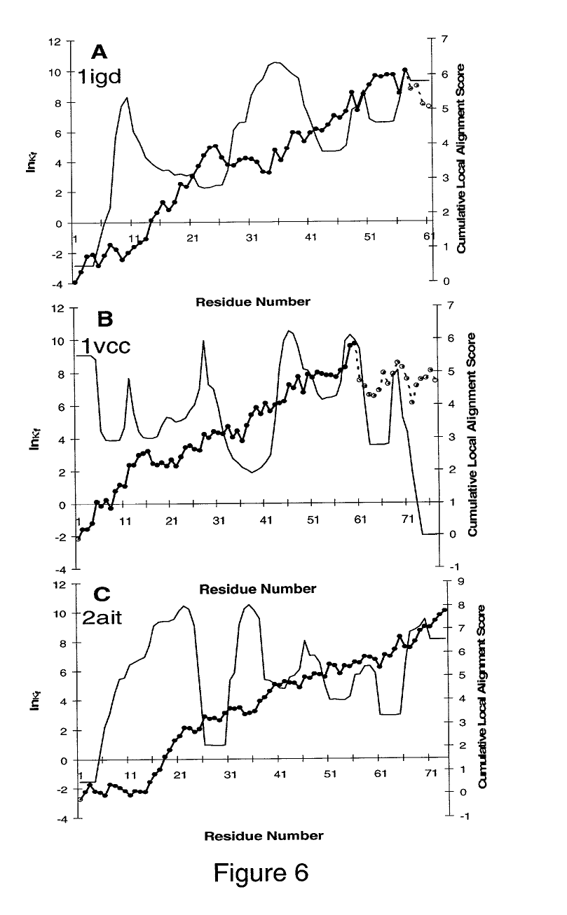

To assess the extent of local alignments that were structurally significant, minor modifications were made to the PROFILESEARCH source code that saved the traceback of the alignment matrix. It was found that for targets scoring poorly in the fold-recognition rankings, local alignments of the corresponding sequence were often not significant. However, sequences that scored in the top two bins were often found to be completely and correctly aligned with their target profiles, even though not all of their residues contributed to the overall score due to the rules of the local algorithm. Three examples of successful alignment based on COREX stability data alone are shown in FIGS. 6A, 6B, 6C and Tables 4A, 4B, 4C for the targets Protein G (1igd), DNA topoisomerase I (1vcc), and tendamistat (2ait), respectively. The alignments calculated using the local algorithm were correct, despite the fact that no sequence information about the target was used, and that only a subset of the amino acid sequence was used in the scoring. In addition, it is noteworthy that the success of these examples is not due to merely a small fragment of the sequence, as the cumulative 3D-1D matrix score steadily increase over the entire length of the sequence.

TABLE 4A
Local Alignment Score of 1igd Sequence to 1igd Stability Profile

| | | | | Cumulative |
|---|---|---|---|---|
| | | | 3D-1D | Local |
| Residue | Residue | Stability | Matrix | Alignment |
| Number | Type* | Environment^{a} | Score^{b} | Score^{c,d} |
| 1 | M | L | 0.02 | 0.02 |
| 2 | T | L | 0.30 | 0.32 |
| 3 | P | L | 0.46 | 0.78 |
| 4 | A | L | 0.05 | 0.83 |
| 5 | V | L | −0.33 | 0.50 |
| 6 | T | L | 0.30 | 0.80 |
| 7 | T | L | 0.30 | 1.10 |
| 8 | Y | M | −0.13 | 0.97 |
| 9 | K | H | −0.29 | 0.68 |
| 10 | L | H | 0.19 | 0.87 |
| 11 | V | M | 0.17 | 1.04 |
| 12 | I | M | 0.12 | 1.16 |
| 13 | N | M | 0.10 | 1.26 |
| 14 | G | L | 0.54 | 1.80 |
| 15 | K | L | 0.22 | 2.02 |
| 16 | T | L | 0.30 | 2.32 |
| 17 | L | L | −0.22 | 2.10 |
| 18 | K | L | 0.22 | 2.32 |
| 19 | G | L | 0.54 | 2.86 |
| 20 | E | L | −0.09 | 2.77 |
| 21 | T | L | 0.30 | 3.07 |
| 22 | T | L | 0.30 | 3.37 |
| 23 | T | L | 0.30 | 3.67 |
| 24 | K | L | 0.22 | 3.89 |
| 25 | A | L | 0.05 | 3.94 |
| 26 | V | L | −0.33 | 3.61 |
| 27 | D | L | −0.20 | 3.41 |
| 28 | A | M | −0.03 | 3.38 |
| 29 | E | M | 0.15 | 3.53 |
| 30 | T | M | 0.05 | 3.58 |
| 31 | A | H | −0.02 | 3.56 |
| 32 | E | H | −0.08 | 3.48 |
| 33 | K | H | −0.29 | 3.19 |
| 34 | A | H | −0.02 | 3.17 |
| 35 | F | H | 0.64 | 3.81 |
| 36 | K | H | −0.29 | 3.52 |
| 37 | Q | H | 0.34 | 3.86 |
| 38 | Y | H | 0.48 | 4.34 |
| 39 | A | H | −0.02 | 4.32 |
| 40 | N | M | −0.25 | 4.07 |
| 41 | D | M | 0.26 | 4.33 |
| 42 | N | M | 0.10 | 4.43 |
| 43 | G | M | −0.05 | 4.38 |
| 44 | V | M | 0.17 | 4.55 |
| 45 | D | M | 0.26 | 4.81 |
| 46 | G | M | −0.05 | 4.76 |
| 47 | V | M | 0.17 | 4.93 |
| 48 | W | H | 0.55 | 5.48 |
| 49 | T | H | −0.52 | 4.96 |
| 50 | Y | H | 0.48 | 5.44 |
| 51 | D | M | 0.26 | 5.70 |
| 52 | D | M | 0.26 | 5.96 |
| 53 | A | M | −0.03 | 5.93 |
| 54 | T | M | 0.05 | 5.98 |
| 55 | K | M | 0.00 | 5.98 |
| 56 | T | H | −0.52 | 5.46 |
| 57 | F | H | 0.64 | 6.10 |
| 58 | T | H | −0.52 | 5.58 |
| 59 | V | H | 0.08 | 5.66 |
| 60 | T | H | −0.52 | 5.14 |
| 61 | E | H | −0.08 | 5.06 |

*One of skill in the art recognizes that the Residue types are listed by the one letter amino acid designation.
^{a}H, M, and L denote high, medium, and low stability as defined in the text and in footnote b of Table 3.
^{b}Value of the 3D-1D scoring matrix corresponding to the results of optimal alignment of the 1igd amino acid sequence given in the “Residue Type” column to the 1igd stability profile given in the “Stability Environment” column. These values are highly similar, but not identical, to the average values given in Table 3A because these values are from the scoring matrix produced when the target protein was removed from the database, as described in the text.
^{c}Sum of all the values in the “3D-1D Matrix Score” column up to and including the indicated residue number. Values in boldface were used by the local alignment algorithm (Smith & Waterman, 1981) to compute the optimal sequence to profile alignment.
^{d}Data in the “Cumulative Local Alignment Score” column was used to generate FIG. 5A.

TABLE 4B
Local Alignment Score of 1vcc Sequence to 1vcc Stability Profile

| | | | | Cumulative |
|---|---|---|---|---|
| | | | 3D-1D | Local |
| Residue | Residue | Stability | Matrix | Alignment |
| Number | Type* | Environment^{a} | Score^{b} | Score^{c,d} |
| 1 | M | H | −0.08 | −0.08 |
| 2 | R | H | 0.30 | 0.22 |
| 3 | A | H | −0.01 | 0.21 |
| 4 | L | H | 0.19 | 0.40 |
| 5 | F | H | 0.66 | 1.06 |
| 6 | Y | M | −0.14 | 0.92 |
| 7 | K | L | 0.19 | 1.11 |
| 8 | D | L | −0.25 | 0.86 |
| 9 | G | L | 0.53 | 1.39 |
| 10 | K | L | 0.19 | 1.58 |
| 11 | L | M | −0.04 | 1.54 |
| 12 | F | H | 0.66 | 2.20 |
| 13 | T | M | 0.00 | 2.20 |
| 14 | D | M | 0.28 | 2.48 |
| 15 | N | M | 0.06 | 2.54 |
| 16 | N | M | 0.06 | 2.60 |
| 17 | F | M | −0.36 | 2.24 |
| 18 | L | M | −0.04 | 2.20 |
| 19 | N | M | 0.06 | 2.26 |
| 20 | P | M | −0.11 | 2.15 |
| 21 | V | M | 0.19 | 2.34 |
| 22 | S | M | −0.19 | 2.15 |
| 23 | D | M | 0.28 | 2.43 |
| 24 | D | M | 0.28 | 2.71 |
| 25 | N | M | 0.06 | 2.77 |
| 26 | P | M | −0.11 | 2.66 |
| 27 | A | M | −0.04 | 2.62 |
| 28 | Y | H | 0.50 | 3.12 |
| 29 | E | M | −0.10 | 3.02 |
| 30 | V | M | 0.19 | 3.21 |
| 31 | L | M | −0.04 | 3.17 |
| 32 | Q | M | −0.04 | 3.13 |
| 33 | H | L | 0.22 | 3.35 |
| 34 | V | L | −0.32 | 3.03 |
| 35 | K | L | 0.19 | 3.22 |
| 36 | I | L | −0.31 | 2.91 |
| 37 | P | L | 0.47 | 3.38 |
| 38 | T | L | 0.32 | 3.70 |
| 39 | H | L | 0.22 | 3.92 |
| 40 | L | L | −0.19 | 3.73 |
| 41 | T | L | 0.32 | 4.05 |
| 42 | D | L | −0.25 | 3.80 |
| 43 | V | M | 0.19 | 3.99 |
| 44 | V | H | 0.06 | 4.05 |
| 45 | V | H | 0.06 | 4.11 |
| 46 | Y | H | 0.50 | 4.61 |
| 47 | E | H | −0.10 | 4.51 |
| 48 | Q | H | 0.34 | 4.85 |
| 49 | T | H | −0.47 | 4.38 |
| 50 | W | H | 0.55 | 4.93 |
| 51 | E | H | −0.10 | 4.83 |
| 52 | E | M | 0.15 | 4.98 |
| 53 | A | M | −0.04 | 4.94 |
| 54 | L | M | −0.04 | 4.90 |
| 55 | T | M | 0.00 | 4.90 |
| 56 | R | M | −0.06 | 4.84 |
| 57 | L | H | 0.19 | 5.03 |
| 58 | I | H | 0.10 | 5.13 |
| 59 | F | H | 0.66 | 5.79 |
| 60 | V | H | 0.06 | 5.85 |
| 61 | G | H | −1.11 | 4.74 |
| 62 | S | M | −0.19 | 4.55 |
| 63 | D | L | −0.25 | 4.30 |
| 64 | S | L | −0.05 | 4.25 |
| 65 | K | L | 0.19 | 4.44 |
| 66 | G | L | 0.53 | 4.97 |
| 67 | R | L | −0.34 | 4.63 |
| 68 | R | H | 0.30 | 4.93 |
| 69 | Q | H | 0.34 | 5.27 |
| 70 | Y | M | −0.14 | 5.13 |
| 71 | F | M | −0.36 | 4.77 |
| 72 | Y | L | −0.73 | 4.04 |
| 73 | G | L | 0.53 | 4.57 |
| 74 | K | L | 0.19 | 4.76 |
| 75 | M | L | 0.04 | 4.80 |
| 76 | H | L | 0.22 | 5.02 |
| 77 | V | L | −0.32 | 4.70 |

*One of skill in the art recognizes that the Residue types are listed by the one letter amino acid designation.
^{a}H, M, and L denote high, medium, and low stability as defined in the text and in footnote b of Table 3.
^{b}Value of the 3D-1D scoring matrix corresponding to the results of optimal alignment of the 1vcc amino acid sequence given in the “Residue Type” column to the 1igd stability profile given in the “Stability Environment” column. These values are highly similar, but not identical, to the average values given in Table 3A because these values are from the scoring matrix produced when the target protein was removed from the database, as described in the text.
^{c}Sum of all the values in the “3D-1D Matrix Score” column up to and including the indicated residue number. Values in boldface were used by the local alignment algorithm (Smith & Waterman, 1981) to compute the optimal sequence to profile alignment.
^{d}Data in the “Cumulative Local Alignment Score” column was used to generate FIG. 5B.

Table 4C
Local Alignment Score of 2ait Sequence to 2ait Stability Profile

| | | | | Cumulative |
|---|---|---|---|---|
| | | | 3D-1D | Local |
| Residue | Residue | Stability | Matrix | Alignment |
| Number | Type* | Environment^{a} | Score^{b} | Score^{c,d} |
| 1 | N | L | −0.21 | −0.21 |
| 1 | N | L | −0.21 | −0.21 |
| 2 | T | L | 0.31 | 0.1 |
| 3 | T | L | 0.31 | 0.41 |
| 4 | V | L | −0.3 | 0.11 |
| 5 | S | L | −0.06 | 0.05 |
| 6 | E | L | −0.11 | −0.06 |
| 7 | P | L | 0.47 | 0.41 |
| 8 | A | M | −0.04 | 0.37 |
| 9 | P | M | −0.1 | 0.27 |
| 10 | S | M | −0.14 | 0.13 |
| 11 | C | M | −0.19 | −0.06 |
| 12 | V | M | 0.18 | 0.12 |
| 13 | T | M | −0.02 | 0.1 |
| 14 | L | M | −0.02 | 0.08 |
| 15 | Y | H | 0.44 | 0.52 |
| 16 | Q | H | 0.34 | 0.86 |
| 17 | S | H | 0.18 | 1.04 |
| 18 | W | H | 0.55 | 1.59 |
| 19 | R | H | 0.27 | 1.86 |
| 20 | Y | H | 0.44 | 2.3 |
| 21 | S | H | 0.18 | 2.48 |
| 22 | Q | H | 0.34 | 2.82 |
| 23 | A | H | −0.02 | 2.8 |
| 24 | D | H | −0.14 | 2.66 |
| 25 | N | M | 0.11 | 2.77 |
| 26 | G | L | 0.53 | 3.3 |
| 27 | C | L | −0.11 | 3.19 |
| 28 | A | L | 0.05 | 3.24 |
| 29 | E | L | −0.11 | 3.13 |
| 30 | T | L | 0.31 | 3.44 |
| 31 | V | M | 0.18 | 3.62 |
| 32 | T | M | −0.02 | 3.6 |
| 33 | V | H | 0.06 | 3.66 |
| 34 | K | H | −0.28 | 3.38 |
| 35 | V | H | 0.06 | 3.44 |
| 36 | V | H | 0.06 | 3.5 |
| 37 | Y | H | 0.44 | 3.94 |
| 38 | E | M | 0.14 | 4.08 |
| 39 | D | M | 0.28 | 4.36 |
| 40 | D | M | 0.28 | 4.64 |
| 41 | T | M | −0.02 | 4.62 |
| 42 | E | M | 0.14 | 4.76 |
| 43 | G | M | −0.04 | 4.72 |
| 44 | L | M | −0.02 | 4.7 |
| 45 | C | M | −0.19 | 4.51 |
| 46 | Y | H | 0.44 | 4.95 |
| 47 | A | M | −0.04 | 4.91 |
| 48 | V | M | 0.18 | 5.09 |
| 49 | A | M | −0.04 | 5.05 |
| 50 | P | M | −0.1 | 4.95 |
| 51 | G | L | 0.53 | 5.48 |
| 52 | Q | M | −0.04 | 5.44 |
| 53 | I | L | −0.34 | 5.1 |
| 54 | T | L | 0.31 | 5.41 |
| 55 | T | M | −0.02 | 5.39 |
| 56 | V | M | 0.18 | 5.57 |
| 57 | G | M | −0.04 | 5.53 |
| 58 | D | M | 0.28 | 5.81 |
| 59 | G | M | −0.04 | 5.77 |
| 60 | Y | M | −0.09 | 5.68 |
| 61 | I | L | −0.34 | 5.34 |
| 62 | G | L | 0.53 | 5.87 |
| 63 | S | L | −0.06 | 5.81 |
| 64 | H | L | 0.3 | 6.11 |
| 65 | G | L | 0.53 | 6.64 |
| 66 | H | M | −0.43 | 6.21 |
| 67 | A | H | −0.02 | 6.19 |
| 68 | R | H | 0.27 | 6.46 |
| 69 | Y | H | 0.44 | 6.9 |
| 70 | L | H | 0.18 | 7.08 |
| 71 | A | H | −0.02 | 7.06 |
| 72 | R | H | 0.27 | 7.33 |
| 73 | C | H | 0.24 | 7.57 |
| 74 | L | H | 0.18 | 7.75 |

*One of skill in the art recognizes that the Residue types are listed by the one letter amino acid designation.
^{a}H, M, and L denote high, medium, and low stability as defined in the text and in footnote b of Table 3.
^{b}Value of the 3D-1D scoring matrix corresponding to the results of optimal alignment of the 2ait amino acid sequence given in the “Residue Type” colunm to the 1igd stability profile given in the “Stability Environment” column. These values are highly similar, but not identical, to the average values given in Table 3A because these values are from the scoring matrix produced when the target protein was removed from the database, as described in the text.
^{c}Sum of all the values in the “3D-1D Matrix Score” column up to and including the indicated residue number. Values in boldface were used by the local alignment algorithm (Smith & Waterman, 1981) to compute the optimal sequence to profile alignment.
^{d}Data in the “Cumulative Local Alignment Score” column was used to generate FIG. 5C.

### Example 10

### State of Ensemble Using COREX

A database of 81 proteins, 5849 residues total (Table 5), was selected from the Protein Data Bank (Baldwin and Rose, 1999) on the basis of biological and computational criteria as described previously in Example 1.

Next, the COREX algorithm (Hilser & Freire, 1996) was run with a window size of five residues on each protein in the database. The minimum window size was set to four, and the simulated temperature was 25° C. The COREX algorithm generated an ensemble of partially unfolded microstates using the high-resolution structure of each protein as a template (Hilser & Freire, 1996) similar to Example 2. This was facilitated by combinatorially unfolding a predefined set of folding units (i.e., residues 1-5 are in the first folding unit, residues 6-10 are in the second folding unit, etc.). By means of an incremental shift in the boundaries of the folding units, an exhaustive enumeration of the partially unfolded species was achieved for a given folding unit size (Hilser & Frieir, 1996; Wrabl, et al., 2001).

Next, the Gibbs free energy for each state, ΔGᵢ relative to the fully-folded reference state was calculated from surface area- and conformational entropy-based parameterizations described previously in Example 2 (Wrabl et al., 2001). Thus, the ΔGᵢ, of each state arises from differences in solvation of apolar and polar surface area, and from differences in conformational entropy between each state and the reference state. Therefore, dividing the free energy into its component terms gives:

ΔGᵢ=ΔG_{apolar,i}+ΔG_{polar,i}+ΔG_{confS,i} (9)

As Equation 9 indicates, different values for the component contributions can provide similar magnitudes for ΔGᵢ, suggesting that different states can have similar stabilities, but different mechanisms for achieving that stability.

TABLE 5
Proteins Used in the COREX Thermodynamic Database

| | | | | | | | | NMR/ | SS_{—} | |
|---|---|---|---|---|---|---|---|---|---|---|
| No. | PDB | Length | SS | SCOP Fold | Score | Z-Score | Rank | X-RAY | CLA | Data Base |
| 1 | 1A1I:A | 85 | Small | Classic zinc finger C2H2 | 6.97 | 1.3613 | 517 | 2.3 | 4 | PDB Select |
| | | | Proteins | | | | | | | |
| 2 | 1A6S:_{—} | 87 | All Alpha | Retroviral matrix protein | 9.04 | 2.8664 | 62 | | 1 | SCOP_files |
| 3 | 1A8O:_{—} | 70 | All alpha | Acyl carrier protein like | 9.26 | 3.7207 | 6 | 1.7 | 1 | Non- |
| | | | | | | | | | | homologous(44) |
| 4 | 1AA3:_{—} | 63 | Alpha + beta | Anti LPS factor/RecA domain | 7.16 | 1.9451 | 62 | NMR | 3 | Non- |
| | | | | | | | | | | homologous(44) |
| 5 | 1ABA:_{—} | 87 | Alpha and | Thioredoxin fold | 12.68 | 5.2207 | 1 | 1.45 | 3 | PDB Select |
| | | | beta | | | | | | | |
| 6 | 1ADR:_{—} | 76 | All alpha | Lambda repressor like DNA | 9.21 | 2.9454 | 53 | NMR | 1 | Non- |
| | | | | binding domains | | | | | | homologous(44) |
| 7 | 1AIW:_{—} | 62 | All Beta | WW domain like | 8.46 | 2.5896 | 53 | | 2 | SCOP_files |
| 8 | 1AN4:A | 65 | All Alpha | helix loop helix DNA binding | 5.58 | 0.6515 | 617 | | 1 | SCOP_files |
| | | | | domain | | | | | | |
| 9 | 1AOI:B | 83 | All Alpha | Histone-fold | 6.21 | 0.7564 | 826 | | 1 | Misc |
| 10 | 1AVY:C | 68 | Coiled coil | Parallel coiled-coil | 7.58 | 2.5997 | 98 | 1.85 | 4 | PDB Select |
| | | | proteins | | | | | | | |
| 11 | 1B9G:A | 57 | Small | Insulin like | 8.53 | 3.5155 | 7 | NMR | 4 | Non- |
| | | | proteins | | | | | | | homologous(44) |
| 12 | 1BDD:_{—} | 60 | All alpha | Bacterial Ig/albumin binding | 10.2 | 4.2561 | 18 | NMR | 1 | Non- |
| | | | | | | | | | | homologous(44) |
| 13 | 1BDO:_{—} | 80 | All beta | Barrel sandwich hybrid | 12.41 | 6.6063 | 1 | 1.8 | 2 | Non- |
| | | | | | | | | | | homologous(44) |
| 14 | 1BF4:A | 63 | Alpha + beta | IL8-like | 9.25 | 3.78 | 7 | 1.6 | 3 | PDB Select |
| 15 | 1BG8:A | 76 | All Alpha | Protein HNS dependent | 6.09 | 0.6685 | 872 | | 1 | SCOP_files |
| | | | | expression A | | | | | | |
| 16 | 1BO9:A | 73 | All Alpha | Annexin | 10.02 | 4.0935 | 5 | | 1 | SCOP_files |
| 17 | 1C1Y:B | 77 | Alpha/beta | P-loop containing NTP | 4.42 | −0.8659 | 3508 | 1.9 | 3 | Non- |
| | | | | hydrolases | | | | | | homologous(44) |
| 18 | 1CC5:_{—} | 83 | All Alpha | Cytochrome C | 10.69 | 4.8891 | 1 | | 1 | Low Sequence |
| | | | | | | | | | | Identity |
| 19 | 1CHC:_{—} | 68 | Small | RING finger domain C3HC4 | 3.98 | −0.4757 | 2617 | NMR | 4 | Non- |
| | | | proteins | | | | | | | homologous(44) |
| 20 | 1CTF:_{—} | 68 | Alpha + beta | Ribosomal protein L7/12 C- | 5.05 | −0.0593 | 2014 | 1.7 | 3 | Non- |
| | | | | terminal fragment | | | | | | homologous(44) |
| 21 | 1CYO:_{—} | 88 | Alpha + beta | Cytochrome b5 | 6.68 | 1.5073 | 379 | 1.5 | 3 | PDB Select |
| 22 | 1D3B:B | 81 | All beta | Sm motif of small nuclear | 11.2 | 5.0183 | 2 | 2 | 2 | PDB Select |
| | | | | ribonucleoproteins, SNRNP | | | | | | |
| 23 | 1DOQ:A | 69 | All Alpha | SAM domain like | 9.68 | 3.8185 | 4 | | | |
| 24 | 1DT4:A | 73 | Alpha + beta | KH domain | 14.45 | 7.5708 | 3 | 2.6 | 3 | Non- |
| | | | | | | | | | | homologous(44) |
| 25 | 1EGW:A | 71 | Alpha + | SRF-like | 6.57 | 1.5595 | 346 | 1.5 | 3 | PDB Select |
| | | | beta | | | | | | | |
| 26 | 1EO0:A | 77 | All Alpha | N cb1 like | 8.89 | 3.7289 | 31 | | 1 | SCOP_files |
| 27 | 1FGP:_ | 70 | All Beta | N-terminal domains of the | 8.64 | 2.3904 | 50 | | 2 | SCOP_files |
| | | | | minor coat protein g3p | | | | | | |
| 28 | 1GDC:_{—} | 72 | Small | Glutacorticoid receptor like | 5.67 | 0.3324 | 1255 | | 4 | Misc |
| | | | proteins | DNA binding domain | | | | | | |
| 29 | 1HCR:A | 52 | All alpha | DNA/RNA-binding 3-helical | 10.28 | 5.0104 | 1 | 1.8 | 1 | PDB Select |
| 30 | 1HDJ:_{—} | 77 | All alpha | Long alpha hairpin | 10.72 | 4.9646 | 2 | | 1 | SCOP_files |
| 31 | 1HOE:_ | 74 | All beta | alpha-Amylase inhibitor | 10.8 | 4.9686 | 5 | 2 | 2 | PDB Select |
| | | | | tendamistat | | | | | | |
| 32 | 1HP8:_{—} | 68 | All alpha | p8-MTCPI | 11.43 | 4.1189 | 13 | | 1 | SCOP_files |
| 33 | 1IIE:A | 75 | All Alpha | MHC class II extoplasmic | 8.8 | 3.4092 | 19 | | 1 | SCOP_files |
| | | | | trimerization domain | | | | | | |
| 34 | 1IRO:_{—} | 53 | Small | Rubredoxin like | 7.57 | 2.422 | 58 | 1.1 | 4 | Non- |
| | | | proteins | | | | | | | homologous(44) |
| 35 | 1ISU:A | 62 | Small | HiPiP | 10.71 | 5.3834 | 1 | 1.5 | 4 | Non- |
| | | | proteins | | | | | | | homologous(44) |
| 36 | 1KDX:A | 81 | All Alpha | Kix domain of CBP | 18.12 | 11.2132 | 1 | | 1 | SCOP_files |
| 37 | 1KJS:_{—} | 74 | All alpha | Anaphylotoxins (complement | 7.29 | 2.0885 | 168 | NMR | 3 | Misc |
| | | | | system) | | | | | | |
| 38 | 1KVE:A | 63 | Alpha + beta | Yeast killer toxins | 6.26 | 1.099 | 443 | 1.8 | 3 | PDB Select |
| 39 | 1KWA:A | 88 | All Beta | PDZ domain like | 9.22 | 2.8257 | 63 | | 2 | SCOP_files |
| 40 | 1MHO:_{—} | 88 | All Alpha | EF Hand-like | 12.97 | 5.5138 | 13 | | 1 | Misc |
| 41 | 1MJC:_{—} | 69 | All beta | OB fold | 8.31 | 2.8477 | 24 | 2 | 2 | Non- |
| | | | | | | | | | | homologous(44) |
| 42 | 1MKN:A | 59 | Small | Midkine | 8.58 | 3.3845 | 20 | NMR | 4 | Non- |
| | | | proteins | | | | | | | homologous(44) |
| 43 | 1MOF:_{—} | 53 | Peptides | MoMLV p15 fragment | 3.47 | −0.4586 | 2558 | 1.7 | 4 | PDB Select |
| | | | | (residues 409-426) | | | | | | |
| 44 | 1MWP:A | 96 | Alpha + | SRCR-like | 5.25 | 0.0759 | 2302 | | 3 | Misc |
| | | | Beta | | | | | | | |
| 45 | 1NHM:_{—} | 79 | All alpha | HMG box | 6 | 0.6857 | 1133 | NMR | 1 | Non- |
| | | | | | | | | | | homologous(44) |
| 46 | 1NKL:_ | 78 | All alpha | Saposin | 10.73 | 4.7036 | 8 | NMR | 1 | Non- |
| | | | | | | | | | | homologous(44) |
| 47 | 1NPS:A | 88 | All Beta | Crystallins proteins yeast killer | 13.77 | 6.2583 | 3 | | 2 | SCOP_files |
| | | | | toxin | | | | | | |
| 48 | 1NRE:_{—} | 81 | All alpha | Alpha 2 macroglobulin receptor | 7.26 | 1.5261 | 355 | | 1 | SCOP_files |
| | | | | associate protein (RAP) domain | | | | | | |
| | | | | I | | | | | | |
| 49 | 1NTC:A | 91 | All Alpha | FIS - like | 7.04 | 1.2342 | 786 | | 1 | SCOP_files |
| 50 | 1NXB:_{—} | 62 | Small | Snake toxin-like | 9.61 | 3.7616 | 31 | 1.38 | 4 | PDB Select |
| | | | Proteins | | | | | | | |
| 51 | 1OPD:_{—} | 85 | Alpha + beta | Histidine-containing | 9.8 | 2.934 | 53 | 1.5 | 3 | PDB Select |
| | | | | phosphocarrier proteins (HPr) | | | | | | |
| 52 | 1OTF:A | 59 | Alpha + beta | Tautomerase/MIF | 8.52 | 3.5069 | 20 | 1.9 | 3 | PDB Select |
| 53 | 1PCF:A | 66 | Alpha + beta | Transcriptional coactivator | 6.05 | 0.4689 | 954 | 1.74 | 3 | PDB Select |
| | | | | PC4 C-terminal domain | | | | | | |
| 54 | 1PGB:_{—} | 56 | Alpha + | beta-Grasp (ubiquitin-like) | 10.12 | 3.9136 | 13 | | 3 | Misc |
| | | | Beta | | | | | | | |
| 55 | 1PLC:_{—} | 99 | All Beta | Cupredoxins | 15.99 | 8.607 | 3 | | 2 | Low Sequence |
| | | | | | | | | | | Identity |
| 56 | 1PTF:_{—} | 87 | Alpha + beta | HPr proteins | 6.95 | 1.4832 | 328 | 1.6 | 3 | Non- |
| | | | | | | | | | | homologous(44) |
| 57 | 1PTQ:_{—} | 50 | Small | Protein kinase (cys2, phorbol- | 6.11 | 1.4565 | 348 | 1.95 | 4 | PDB Select |
| | | | Proteins | binding domain) | | | | | | |
| 58 | 1PTX:_ | 64 | Small | Knottins | 11.22 | 4.9044 | 9 | 1.3 | 4 | Non- |
| | | | proteins | | | | | | | homologous(44) |
| 59 | 1QA4:A | 56 | Peptides | HIV-1 Nef protein fragments | 3.66 | −0.1927 | 1886 | NMR | 4 | Non- |
| | | | | | | | | | | homologous(44) |
| 60 | 1QGW:B | 67 | Alpha + beta | Non-globular alpha beta | 6.42 | 1.6833 | 252 | 1.63 | 3 | PDB Select |
| | | | | subunits of globular proteins | | | | | | |
| 61 | 1QQV:A | 67 | All alpha | Thermostable subdomain from | 7.49 | 2.2425 | 114 | NMR | 1 | Non- |
| | | | | chicken villin | | | | | | homologous(44) |
| 62 | 1R1B:A | 56 | All alpha | SIS/NS1 RNA binding domain | 5.47 | 0.3507 | 1035 | NMR | 1 | Non- |
| | | | | | | | | | | homologous(44) |
| 63 | 1ROP:_{—} | 56 | All alpha | ROP-like | 6.76 | 1.5118 | 307 | | 1 | Misc |
| 64 | 1RZL:_{—} | 91 | All Alpha | Bifunctional inhibitor/lipid- | 14.93 | 7.4941 | 1 | | 1 | Misc |
| | | | | transfer protein | | | | | | |
| 65 | 1SHG:_{—} | 57 | All beta | SH3-like barrel | 12.01 | 6.0485 | 6 | | 2 | Misc |
| 66 | 1SKN:P | 74 | All Alpha | Binding domain of skn-1 | 13.97 | 7.0584 | 1 | | 1 | SCOP_files |
| 67 | 1SVF:B | 62 | Coiled coil | Stalk segment of viral fusion | 4.63 | 0.8663 | 503 | 1.4 | 4 | PDB Select |
| | | | proteins | proteins | | | | | | |
| 68 | 1TBA:A | 67 | All alpha | TAF II 230 nTBP binding | 6.39 | 1.5715 | 243 | | 1 | SCOP_files |
| | | | | fragment | | | | | | |
| 69 | 1TGS:I | 56 | Small | Ovomucoid/PCI-1 like | 8.3 | 2.9194 | 73 | | 4 | Low Sequence |
| | | | proteins | inhibitors | | | | | | Identity |
| 70 | 1TRL:A | 62 | All alpha | Thermolysin like metallo- | 7.23 | 1.6302 | 317 | | 1 | SCOP_files |
| | | | | proteases C-terminal domain | | | | | | |
| 71 | IUGI:D | 82 | Alpha + beta | Cystatin-like | 13.45 | 5.7713 | 16 | 1.55 | 3 | PDB Select |
| 72 | 1UTG:_{—} | 70 | All alpha | Uteroglobin-like | 10.81 | 5.0354 | 1 | 1.34 | 1 | PDB Select |
| 73 | 1VCC:_{—} | 77 | Alpha + beta | DNA topoisomerase I domain | 11.17 | 5.0864 | 2 | 1.6 | 3 | Non- |
| | | | | | | | | | | homologous(44) |
| 74 | 2ABD:_{—} | 86 | All Alpha | acyl CoA binding protein like | 9.48 | 3.1371 | 33 | | | |
| 75 | 2BOP:A | 85 | Alpha + beta | Ferredoxin-like | 13.38 | 6.3446 | 1 | 1.7 | 3 | PDB Select |
| 76 | 2CI2:I | 65 | Alpha + beta | C12 family of serine protease | 8.12 | 2.4352 | 41 | 2 | 3 | Non- |
| | | | | inhibitors | | | | | | homologous(44) |
| 77 | 2KNT:_{—} | 58 | Small | BPTI-like | 20.54 | 9.4078 | 3 | 1.2 | 4 | PDB Select |
| | | | Proteins | | | | | | | |
| 78 | 2SPG:A | 66 | All beta | Beta clip | 11.24 | 4.341 | 3 | 1.75 | 2 | Non- |
| | | | | | | | | | | homologous(44) |
| 79 | 3EIP:A | 84 | Alpha + | FKBP-like | 10.98 | 4.8841 | 6 | 1.8 | 3 | PDB Select |
| | | | beta | | | | | | | |
| 80 | 3NCM:A | 92 | All Beta | Immunoglobulin-like beta | 13.15 | 6.45 | 1 | | 2 | SCOP_files |
| | | | | sandwhich | | | | | | |
| 81 | 5HPG:A | 84 | Small | Kringle-like | 15.08 | 7.9213 | 1 | 1.66 | 4 | PDB Select |
| | | | Proteins | | | | | | | |

### Example 11

### Surface Area Calculations

The calorimetric enthalpy and entropy of solvation were parameterized from polar and apolar surface exposure (Hilser & Freire, 1996). COREX uses empirical parameterizations to calculate the relative apolar and polar free energies of each microstate:

ΔG_{apolar,i}(T)=−8.44*ΔASA_{apolar,i}+0.45*ΔASA_{apolar,i}*(T−333)−T*(045*ΔASA_{apolar,i}*ln(T/385)) (10)

ΔG_{polar,i}(T)=31.4.44*ΔASA_{polar,i}−0.26*ΔASA_{polar,i}*(T−333)−T*(−0.26*ΔASA_{polar,i}*ln(T/335)) (11)

The three primary components used to calculate conformational entropies (ΔS_{i,conf}) for each microstate were: (1) ΔS_{bu→ex}, the entropy change associated with the transfer of a side-chain that was buried in the interior of the protein to its surface; (2) ΔS_{ex→u}, the entropy change gained by a surface-exposed side-chain when the peptide backbone unfolds; and (3) ΔS_{bb}, the entropy change gained by the backbone itself upon unfolding (Hilser & Freire, 1996). For fold recognition calculations, the total (ΔS_{i,conf}) of all proteins is multiplied by a scaling factor to eliminate the unfolded state contribution to the residue-specific thermodynamic parameters.

Next, the residue stability constant, κ_{f}, was calculated similar to Example 2. The residue stability constant is the ratio of the summed probability of all states in the ensemble in which a particular residue, j, is in a folded conformation (ΣP_{f,j}) to the summed probability of all states in which residue j is in an unfolded (i.e., non-folded) conformation (ΣP_{nf,j}).

Equation 2, in turn, was used to define a residue-specific free energy of folding for the protein (ΔG_{f,j}=−RTlnκ_{f,j}), which was expanded to give (ΔG_{f,j}=RT lnQ_{nf,j}−RT ln Q_{f,j}) where Q_{nf,j} and Q_{f,j} were the sub-partition functions for states in which residue j was unfolded and folded, respectively. Thus, the residue-specific free energy provides the difference in energy between the sub-ensembles in which each residue is folded and unfolded. In other words, the residue stability constant does not provide the contribution of each amino acid to the stability of a protein. Rather, it provides the relative stability of that region of the protein, implicitly considering the contribution of all amino acids in the protein toward the observed stability at that position.

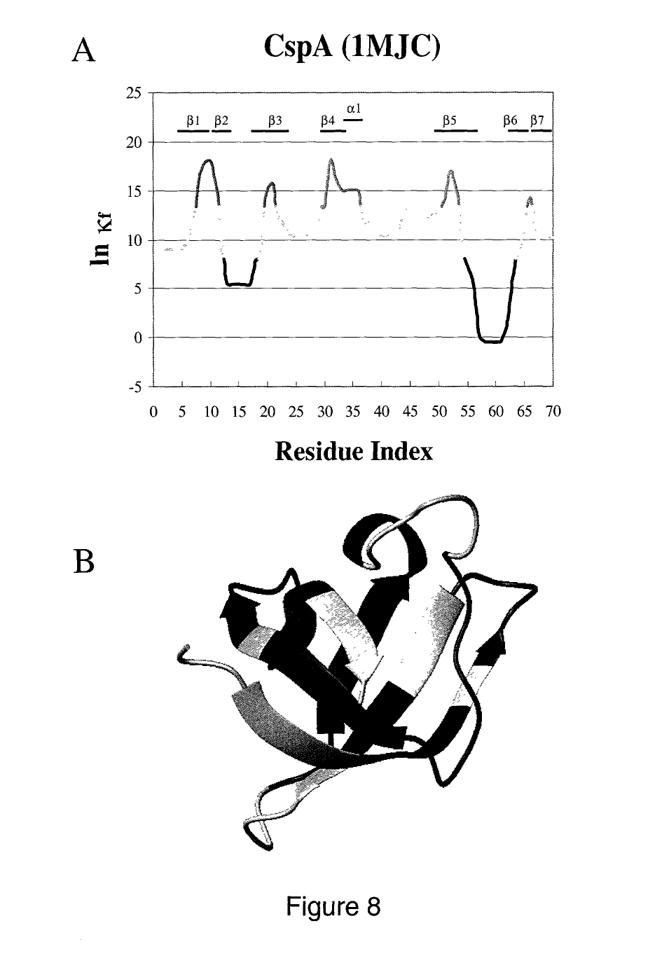

As shown in FIG. 8, the stability constants provided a residue-specific description of the regional differences in stability within a protein structure. The importance of this quantity from the point of view of fold recognition is two-fold. First, the stability constant is compared directly to protection factors obtained from native state hydrogen exchange experiments, thus providing an experimentally verifiable residue-specific description of the ensemble. Second, as amino acids are non-randomly distributed across high, medium and low stability environments, the stability constant as a function of residue position provides a convenient 1-dimensional representation of the 3-dimensional structure.

### Example 12

### Identification of Additional Thermodynamic Determinants

First, the ΔGᵢ for each microstate i in the ensemble was composed of solvation and conformational entropy terms as described by Equation 9 and Example 10. Equation 9 was rewritten in terms of the enthalpic and entropic components:

ΔGᵢ=ΔH_{i,solvation}−T(ΔS_{i,solvation}+ΔS_{i,conformational}) (12)

Each of the solvation terms in Equation 12 was further expanded into contributions based on apolar and polar surface area:

ΔGᵢ=(ΔH_{i,solvation,apolar}+ΔH_{i,solvation,polar})−T(ΔS_{i,solvation,apolar}+ΔS_{i,solvation,polar})−T(ΔS_{i,conformational}) (13)

However, the identical values for the apolar and polar areas of each state were used for the respective terms in the enthalpy and entropy calculations. Therefore, the absolute values for the enthalpy and entropy terms for a given area type were related by constants k₁ (for apolar area) and k₂ (for polar area), yielding the expression:

ΔGᵢ=(ΔH_{solvation,apolar}+ΔH_{solvation,polar})−T(k₁ΔH_{i,solvation,apolar}+k₂ΔH_{i,solvation,polar})−T(ΔS_{i,conformational}) (14)

Grouping area types together and simplifying gives:

ΔGᵢ=[(ΔH_{i,solvation,apolar})*(1−T*k₁)]+[(ΔH_{i,solvation,apolar})*(1−T*k₂)]−T(ΔS_{i,conformational}) (15)

Equation 15 revealed that for a given free energy and conformational entropy, the relative contribution of polar and apolar surface to the solvation free energy was ascertained from the ratio of polar to apolar enthalpy for each state.

Thus, to arrive at a residue-specific contribution of polar and apolar solvation, a given thermodynamic parameter (i.e. enthalpy or entropy) is considered an average excess quantity, which represents the population-weighted contribution of all states in the ensemble. For instance, the average excess enthalpy and entropy was defmed as:

$$\begin{matrix}⟨ Δ H ⟩ = {\sum }_{i = 1}^{{N}_{states}} {P}_{i} \cdot Δ {H}_{i} = {\sum }_{i = 1}^{{N}_{states}} \frac{{K}_{i} \cdot Δ {H}_{i}}{Q} . & \text{(16A)} \\ ⟨ Δ S ⟩ = {\sum }_{i = 1}^{{N}_{states}} {P}_{i} \cdot Δ {S}_{i} = {\sum }_{i = 1}^{{N}_{states}} \frac{{K}_{i} \cdot Δ {S}_{i}}{Q} & \text{(16B)}\end{matrix}$$

Following from Equations 16A and 16B, residue-specific descriptors of the polar and apolar enthalpy were defined accordingly. The polar component of the enthalpy was defined as the difference between the average excess polar enthalpy from the sub-ensemble in which residue j is folded (<ΔH_{pol,f,j}>) and the average excess polar enthalpy from the sub-ensemble in which residue j is unfolded (<ΔH_{pol,nf,j}>):

ΔH_{pol,j}=<ΔH_{pol,f,j}>−<ΔH_{pol,nf,j>} (17)

where:

$$\begin{matrix}⟨ Δ {H}_{pol , f , j} ⟩ = {\sum }_{i = 1}^{N j , folded} ( \frac{( Δ {H}_{pol , f , i} \cdot {ⅇ}^{- Δ {G}_{i} / RT} )}{{Q}_{f , j}} ) & ( 18 ) \\ ⟨ Δ {H}_{pol , nf , j} ⟩ = {\sum }_{i = 1}^{N j , not folded} ( \frac{( Δ {H}_{pol , nf , i} \cdot {ⅇ}^{- Δ {G}_{i} / RT} )}{{Q}_{nf , j}} ) & ( 19 )\end{matrix}$$

It is important to note that the summations in Equations 18 and 19 were only over the sub-ensembles in which residue j was folded and unfolded, respectively, and the parameters Q_{f,j} and Q_{nf,j} were the sub-partition functions for those sub-ensembles. By identical reasoning, the residue-specific apolar component to the enthalpy of residue j and the residue-specific conformational entropy component of residue j were defined as:

ΔH_{apol,j}<=ΔH_{apol,f,j}>−<ΔH_{apol,nf,j>} (20)

ΔS_{conf,j}=<ΔS_{conf,j}>−<ΔS_{conf,nf,j>} (21)

As in the case with the residue stability constant, the expressions for the residue-specific ΔH_{apol,j}, ΔH_{pol,j} and ΔS_{conf,j} do not provide the contributions of residue j to the respective overall thermodynamic properties. Instead, Equations 17, 20 and 21 reflect the average thermodynamic environments of that residue, accounting implicitly for the contribution of all the amino acids over all the states in the ensemble.

### Example 13

### Residue-Specific Thermodynamic Environments

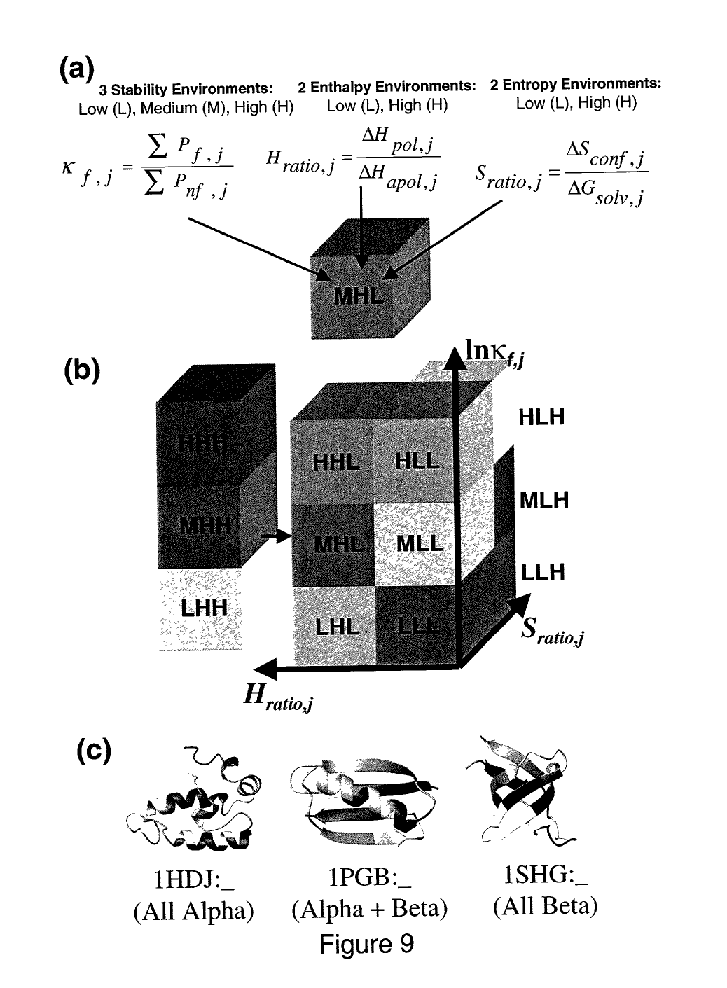

Using Equations 2, 17, 20, and 21, thermodynamic environments were empirically defined so as to systematically account for the different contributions of solvation and conformational entropy to the overall stability constant of each residue. As shown in FIG. 9A-FIG. 9C, three thermodynamic dimensions were considered; stability (κ_{f,j}), enthalpy (H_{ratio,j}), and entropy (S_{ratio,j}). The first dimension utilizes the stability constant classification (FIG. 8A and FIG. 8B) defined by Equation 2. As the particular value for the stability constant can arise from conformational entropy or solvent related phenomena, a second dimension was utilized that provided the ratio of the conformational entropy to the total solvation free energy;

$${S}_{ratio , j} = \frac{Δ {S}_{conf , j}}{Δ {G}_{solv , j}} \qquad ( 22 )$$

where ΔG_{solv,j} is the total residue-specific solvation component calculated similar to Equations 17-21. Finally, as the total solvation component can arise from polar or apolar contributions, a third dimension was incorporated that provided the ratio of polar to apolar enthalpy described by Equations 17 and 20;

$${H}_{ratio , j} = \frac{Δ {H}_{pol , j}}{Δ {H}_{apol , j}} \qquad ( 23 )$$

Thus, the residues making up the 81 proteins (Table 5) that were analyzed partitioned non-randomly within the three-dimensional thermodynamic space. The non-random distribution of residues resulted in an empirical partitioning of the residue-specific data into twelve thermodynamic categories by dividing the stability data into three categories, the enthalpy data into two categories, and the entropy data into two categories (FIG. 9A-FIG. 9C).

### Example 14

### Binning of Thermodynamic Environments

Each of the 5849 residues in the database were binned into one of the twelve thermodynamic environment classes based on their stability (κ_{f,j}), enthalpy (H_{ratio,j}), and entropy (S_{ratio,j}) values. These thermodynamic environments were denoted by the following abbreviations: LLL, LLH, LHL, LHH, MLL, MLH, MHL, MHH, HLL, HLH, HHL, HHH. For example, residues in the LMH thermodynamic environment were binned into the Low (L) stability (κ_{f,j}) class, the Medium (M) enthalpy (H_{ratio,j}) class, and the High (H) entropy (S_{ratio,j}) class. The cutoffs for each thermodynamic class were defined as:

Stability (κ_{f,j}) class (L, M, or H):

Low κ_{f,j} (L)≡[lnκ_{f,j}<7.95] (22)

Medium κ_{f,j} (M)≡[7.95<=lnκ_{f,j}<13.4] (23)

High κ_{f,j} (H)≡[13.4<=lnκ_{f,j]} (24)

Enthalpy (H_{ratio,j}) class (L or H):

Low H_{ratio,j} (L)≡[ΔHₚₒₗ<−1.024*ΔHₐₚ−2553] (25)

High H_{ratio,j} (H)≡[ΔHₚₒₗ>=−1.024*ΔHₐₚ−2553] (26)

Entropy (S_{ratio,j}) class (L or H):

Low S_{ratio,j} (L)≡[−TΔS_{conf}<0.125*ΔGₛₒₗᵥ−3053] (27)

High S_{ratio,j} (H)≡[−TΔS_{conf}>=0.125*ΔGₛₒₗᵥ−3053] (28)

Visual inspection of the segregation of amino acid types as a function of various thermodynamic parameters extracted from the 81-protein COREX database, guided by the development outlined above, suggested that the general classifications of stability, enthalpy, and entropy was reasonably divided thermodynamic space (as indicated in FIG. 9). The exact cutoffs for the twelve residue-specific thermodynamic environments used in the threading calculations were determined automatically by an exhaustive grid search of all possible. The utility of each trial set of cutoffs was initially determined from a coarse search of cutoff space by threading a constant subset of 8 targets in the protein database and recording sets of cutoffs that maximized the Z-scores and percentiles for each target. Then, a finer grid search over the best sets of cutoffs, threading against a subset of 20 targets for each trial set of cutoffs, resulted in the optimized set of cutoffs used for the threading experiments shown in this work. Identical cutoffs were used for the alpha/beta threading calculations, i.e. no special optimization was performed for the scoring of the alpha/beta experiment.

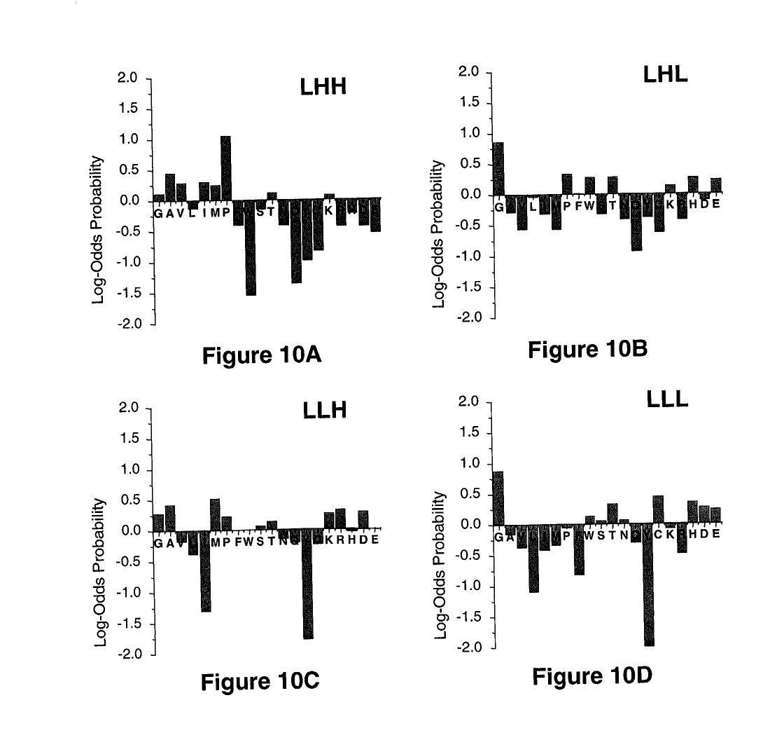

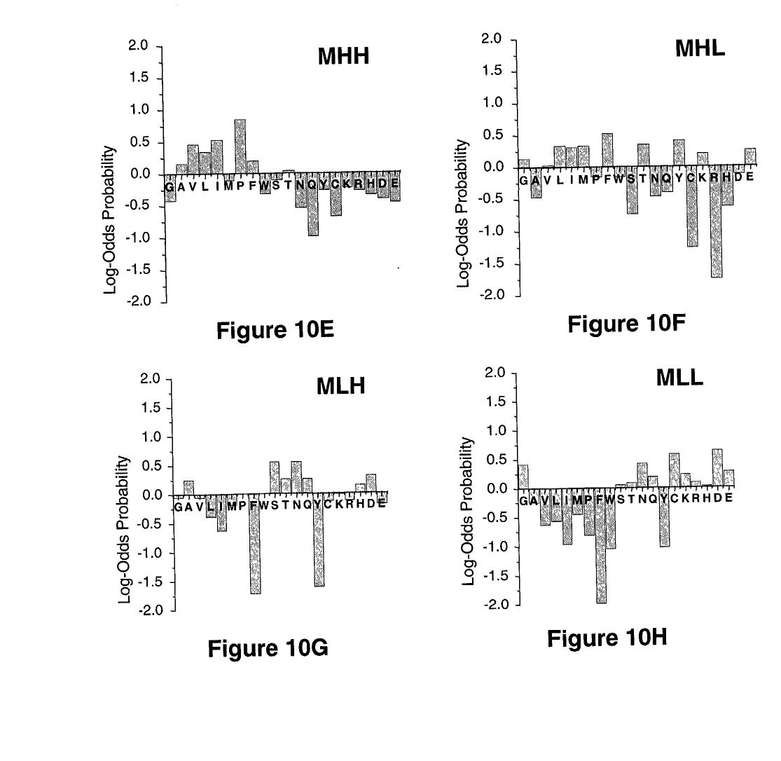

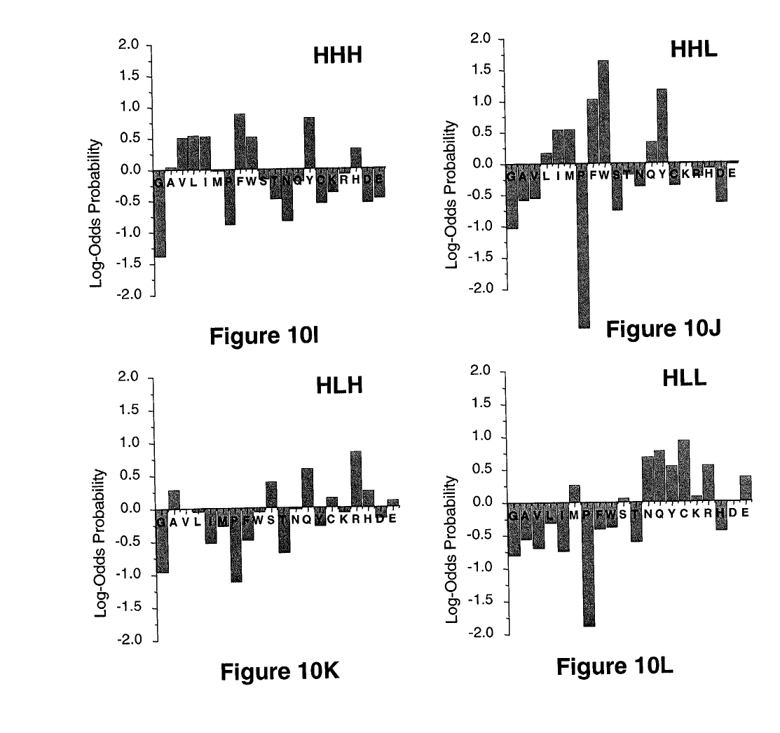

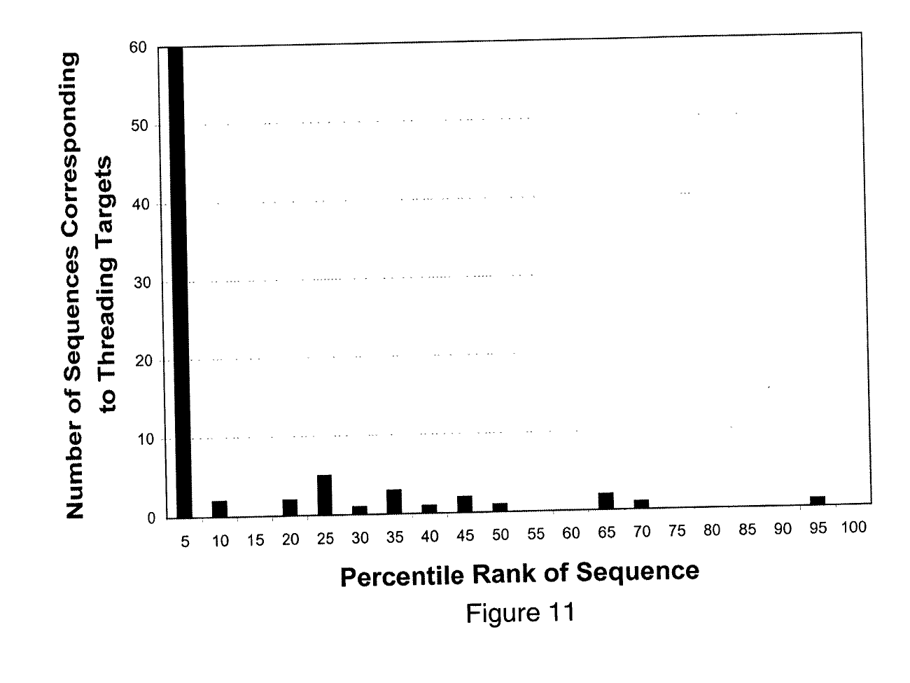

Statistics for amino acid type as a function of each of the thermodynamic environments were tabulated (Table 6) and the log-odds probability for an amino acid type to be in each thermodynamic environment was calculated. The resulting histograms (FIG. 10) revealed a non-random distribution of the amino acids within the thermodynamic environments. For example, hydrophobic residues such as Ile, Phe, and Val were observed with lower frequency in the MLL environment, while polar and charged amino acids such as Asp, Gln, and Lys were observed with higher frequency in this environment. These distributions cannot always be rationalized on the basis of side chain chemical properties, however, as the basic amino acids Arg and Lys exhibited very different propensities to occur in the MHL environment. This latter observation must be a reflection of the fact that ensemble-derived energetics included averaged tertiary enthalpic and entropic information that is not encoded by individual side chain properties alone.

TABLE 6
Statistics of Amino Acid Type as a Function of the Twwelve Thermodynamic
Environments

| | LHH | LHL | LLH | LLL | MHH | MHL | MLH | MLL | HHH | HHL | HLH | HLL | SUM |
|---|---|---|---|---|---|---|---|---|---|---|---|---|---|
| ALA | 48 | 19 | 26 | 30 | 74 | 24 | 55 | 44 | 53 | 15 | 56 | 22 | 466 |
| ARG | 12 | 10 | 14 | 13 | 29 | 4 | 23 | 37 | 28 | 13 | 58 | 40 | 281 |
| ASN | 11 | 9 | 8 | 20 | 20 | 13 | 40 | 46 | 12 | 10 | 22 | 41 | 252 |
| ASP | 14 | 16 | 16 | 32 | 30 | 24 | 41 | 75 | 21 | 10 | 25 | 27 | 331 |
| CYS | 5 | 5 | 5 | 20 | 12 | 4 | 14 | 37 | 11 | 7 | 18 | 36 | 174 |
| GLN | 4 | 5 | 7 | 13 | 12 | 13 | 28 | 34 | 21 | 19 | 38 | 42 | 236 |
| GLU | 17 | 30 | 16 | 42 | 38 | 47 | 34 | 70 | 30 | 25 | 44 | 53 | 446 |
| GLY | 32 | 55 | 21 | 77 | 39 | 41 | 38 | 79 | 12 | 9 | 15 | 16 | 434 |
| HIS | 6 | 8 | 4 | 12 | 11 | 5 | 12 | 14 | 17 | 6 | 13 | 6 | 114 |
| ILE | 27 | 12 | 3 | 15 | 70 | 34 | 15 | 14 | 56 | 30 | 16 | 12 | 304 |
| LEU | 28 | 25 | 12 | 12 | 92 | 55 | 30 | 33 | 91 | 33 | 41 | 29 | 481 |
| LYS | 36 | 31 | 24 | 35 | 55 | 51 | 46 | 76 | 38 | 29 | 42 | 45 | 508 |
| MET | 11 | 4 | 8 | 7 | 16 | 15 | 10 | 10 | 14 | 13 | 9 | 14 | 131 |
| PHE | 8 | 10 | | 6 | 30 | 25 | 3 | 3 | 48 | 29 | 10 | 10 | 182 |
| PRO | 45 | 18 | 11 | 17 | 76 | 17 | 22 | 13 | 11 | 1 | 7 | 3 | 241 |
| SER | 19 | 13 | 13 | 26 | 41 | 13 | 53 | 42 | 31 | 9 | 44 | 29 | 333 |
| THR | 23 | 22 | 13 | 32 | 44 | 36 | 37 | 41 | 21 | 15 | 14 | 14 | 312 |
| TRP | 1 | 5 | | 6 | 7 | 5 | | 3 | 13 | 21 | 6 | 4 | 71 |
| TYR | 4 | 6 | 1 | 1 | 17 | 20 | 3 | 7 | 40 | 30 | 11 | 23 | 163 |
| VAL | 34 | 12 | 12 | 20 | 84 | 33 | 34 | 25 | 71 | 13 | 35 | 16 | 389 |
| SUM | 385 | 315 | 214 | 436 | 797 | 479 | 538 | 703 | 639 | 337 | 524 | 482 | 5849 |

### Example 15

### Fold-Recognition Details

Simple fold-recognition experiments were performed based on amino acid distributions within the twelve thermodynamic environments.

Briefly, a profiling method was used to create thermodynamic environment profiles for each of the 81 proteins in the database (Bowie et al., 1991; Gribskov et al., 1987). The 81 amino acid sequences (Table 5) coding for the native structures used in the database (in addition to 3777 decoy sequences) were each threaded against the 81 target thermodynamic environment profiles. The decoy sequences were obtained from the Protein Data Bank and were inclusive for all sequences coding for “foldable” proteins ranging from 35 to 100 residues.

Next, a 3D-1D scoring matrix for each protein in the database was calculated, in which the scoring matrix data was simply the log-odds probabilities of finding amino acid types in one of the thermodynamic environment classes (Equation 30, below). The resulting profile of the target protein was then optimally aligned to each member of a library of amino acid sequences (i.e. 3858 decoy sequences) by maximizing the score between the sequence and the profile using a local alignment algorithm based on the Smith-Waterman algorithm (Smith & Waterman, 1981) as implemented in PROFILESEARCH (Bowie et al., 1991). No attempt was made to optimize the gap opening and extension penalties for the local algorithm; in all cases these were the default values given in the PROFILESEARCH package, 5.00 and 0.05, respectively. Z-scores were computed from PROFILESEARCH for each threading result from Equation (30):

Z=(S−σ)/<S> (30)

In Equation 30, s was the PROFILESEARCH threading score of a sequence i when threaded against the structure corresponding to sequence i, <S> was the average threading score of all sequences in the database (identical in length to sequence i) threaded against the structure corresponding to sequence i, and σ was the standard deviation of the scores of all sequences in the database (identical in length to sequence i) threaded against the structure corresponding to sequence i. Thus, the Z-score was the number of standard deviations above the mean that sequence i scored against its target.

Nearly three-fourths (60/81) of the correct sequences scored in the top 5^{th} percentile when threaded against their corresponding thermodynamic environment profile (FIG. 10), and the Z-scores (the number of standard deviations a particular sequence scored above the mean score of all chains of identical length) for these successful threadings ranged from 1.76 to 12.23 (Table 7).

TABLE 7
Fold Recognition Results

| No. | PDB | % Rank | Z_SCORE |
|---|---|---|---|
| 1 | 1A1I:A | 0.29 | 3.49 |
| 2 | 1A6S:_{—} | 0.67 | 3.23 |
| 3 | 1A8O:_{—} | 0.34 | 3.29 |
| 4 | 1AA3:_{—} | 3.84 | 2.08 |
| 5 | 1ABA:_{—} | 0.03 | 4.1 |
| 6 | 1ADR:_{—} | 0.93 | 3.71 |
| 7 | 1AIW:_{—} | 2.36 | 2.27 |
| 8 | 1AN4:A | 23.64 | 0.68 |
| 9 | 1AOI:B | 26.31 | 0.52 |
| 10 | 1AVY:C | 5.16 | 1.82 |
| 11 | 1B9G:A | 0.18 | 4.48 |
| 12 | 1BDD:_{—} | 0.44 | 5.07 |
| 13 | 1BDO:_{—} | 0.05 | 6.25 |
| 14 | 1BF4:A | 0.16 | 4.04 |
| 15 | 1BG8:A | 33.23 | 0.32 |
| 16 | 1BO9:A | 0.21 | 4.06 |
| 17 | 1CIY:B | 95.44 | −1.46 |
| 18 | 1CC5:_{—} | 0.13 | 5.3 |
| 19 | 1CHC:_{—} | 67.88 | −0.55 |
| 20 | 1CTF:_{—} | 32.17 | 0.22 |
| 21 | 1CYO:_{—} | 5.47 | 1.76 |
| 22 | 1D3B:B | 0.93 | 2.7 |
| 23 | 1DOQ:A | 0.03 | 4.34 |
| 24 | 1DT4:A | 0.08 | 6.83 |
| 25 | 1EGW:A | 4.33 | 2.14 |
| 26 | 1EO0:A | 0.88 | 4.01 |
| 27 | 1FGP:_{—} | 2.13 | 2.65 |
| 28 | 1GDC:_{—} | 64.41 | −0.45 |
| 29 | 1HCR:A | 0.16 | 4.7 |
| 30 | 1HDJ:_{—} | 1.35 | 2.8 |
| 31 | 1HOE:_{—} | 0.13 | 5.62 |
| 32 | 1HP8:_{—} | 0.47 | 4.43 |
| 33 | 1IIE:A | 0.39 | 3.28 |
| 34 | 1IRO:_{—} | 0.13 | 5.4 |
| 35 | 1ISU:A | 0.54 | 3.58 |
| 36 | 1KDX:A | 0.03 | 9.34 |
| 37 | 1KJS:_{—} | 32.4 | 0.26 |
| 38 | 1KVE:A | 2.41 | 2.5 |
| 39 | 1KWA:A | 0.29 | 3.7 |
| 40 | 1MHO:_{—} | 0.39 | 3.54 |
| 41 | 1MJC:_{—} | 4.07 | 1.99 |
| 42 | 1MKN:A | 3.24 | 2.33 |
| 43 | 1MOF:_{—} | 65.34 | −0.47 |
| 44 | 1MWP:A | 24.29 | 0.56 |
| 45 | 1NHM:_{—} | 17.26 | 0.93 |
| 46 | 1NKL:_{—} | 0.91 | 3.19 |
| 47 | 1NPS:A | 0.13 | 4.36 |
| 48 | 1NRE:_{—} | 24.29 | 0.54 |
| 49 | 1NTC:A | 39.71 | 0.1 |
| 50 | 1NXB:_{—} | 0.78 | 4.1 |
| 51 | 1OPD:_{—} | 4.15 | 2.09 |
| 52 | 1OTF:A | 1.09 | 3.49 |
| 53 | 1PCF:A | 40.95 | 0.17 |
| 54 | 1PGB:_{—} | 0.13 | 5.9 |
| 55 | 1PLC:_{—} | 0.13 | 8.42 |
| 56 | 1PTF:_{—} | 7.34 | 1.63 |
| 57 | 1PTQ:_{—} | 9.62 | 1.33 |
| 58 | 1PTX:_{—} | 0.47 | 4.21 |
| 59 | 1QA4:A | 45.59 | −0.05 |
| 60 | 1QGW:B | 2.95 | 2.25 |
| 61 | 1QQV:A | 1.87 | 2.73 |
| 62 | 1RIB:A | 22.76 | 0.68 |
| 63 | 1ROP:_{—} | 42.48 | 0.02 |
| 64 | 1RZL:_{—} | 0.05 | 6.57 |
| 65 | 1SHG:_{—} | 0.08 | 6.09 |
| 66 | 1SKN:P | 0.03 | 6.28 |
| 67 | 1SVF:B | 20.14 | 0.67 |
| 68 | 1TBA:A | 1.09 | 2.68 |
| 69 | 1TGS:I | 2.62 | 2.6 |
| 70 | 1TRL:A | 23.54 | 0.53 |
| 71 | 1UGI:D | 0.44 | 7.02 |
| 72 | 1UTG:_{—} | 0.08 | 5.92 |
| 73 | 1VCC:_{—} | 0.08 | 4.48 |
| 74 | 2ABD:_{—} | 0.23 | 3.96 |
| 75 | 2BOP:A | 0.03 | 7.09 |
| 76 | 2CI2:I | 5.44 | 2.06 |
| 77 | 2KNT:_{—} | 0.08 | 12.23 |
| 78 | 2SPG:A | 0.39 | 5.31 |
| 79 | 3EIP:A | 0.18 | 5.53 |
| 80 | 3NCM:A | 0.44 | 4.24 |
| 81 | 5HPG:A | 0.05 | 11.02 |

### Example 16

### Construction of Scoring Matrices

The scoring matrices were calculated as log-odds probabilities of finding residue type j in structural environment k, as described below (Wrabl et al., 2001; Bowie et al., 1991). The matrix score, S_{j,k}, was defined as:

$${S}_{j , k} = ln \frac{{P}_{j \vert k}}{{P}_{k}} \qquad ( 27 )$$

P_{j|k} is the probability of finding a residue of type j in stability class k (i.e. number of counts of residue type j in stability class k divided by the total number of counts of residue type j), and Pₖ is the probability of finding any residue in the database in stability environment k (i.e. number of residues in stability class k, regardless of amino acid type, divided by the total number of residues in the entire database, regardless of amino acid type). The structural environment used was one of the twelve COREX thermodynamic environments (LHH, LHL, LLH, LLL, MHH, MHL, MLH, MLL, HHH, HHL, HLH, HLL), as described above. The fold recognition target was removed from the database, and the remaining 80 proteins were used to calculate the probabilities. Therefore, information about the target was never included in the scoring matrix.

### Example 17

### Thermodynamic Information is more Fundamental than Secondary Structure Information

Secondary structure, although useful in the analysis and classification of protein folds, is an easily reportable observable that does little to explain the underlying physical chemistry of protein structure. In fact, secondary structure can be viewed as a manifestation of the backbone/side-chain van der Waals' repulsions that divide phi/psi space, modified by the thermodynamic stability afforded by local and tertiary interactions such as hydrogen bonding and the hydrophobic effect (Srinivasan & Rose, 1999; Baldwin & Rose, 1999). Any reasonable description of the energetics of protein structure must be able to reflect these realities independent of secondary structural propensities of amino acids and the secondary structural classifications of folds.

Although the COREX energy function accounts for specific interactions only in an implicit way, the results of a COREX calculation may provide deeper insight than secondary structure into the structural determinants of protein folds. For example, FIG. 9C compared the thermodynamic environment profiles for an all-alpha protein and an all-beta protein threaded over their native folds. Visual inspection of the two color-coded structures revealed that different thermodynamic environments span single types of secondary structure, and that the same thermodynamic environment was found in different types of secondary structural elements.

Thus, a threading procedure was repeated on a subset of proteins from the original database (Table 5), sorted by secondary structure to determine the possibility that the thermodynamic environments calculated by COREX represented a fundamental property of proteins that transcended structural classifications.

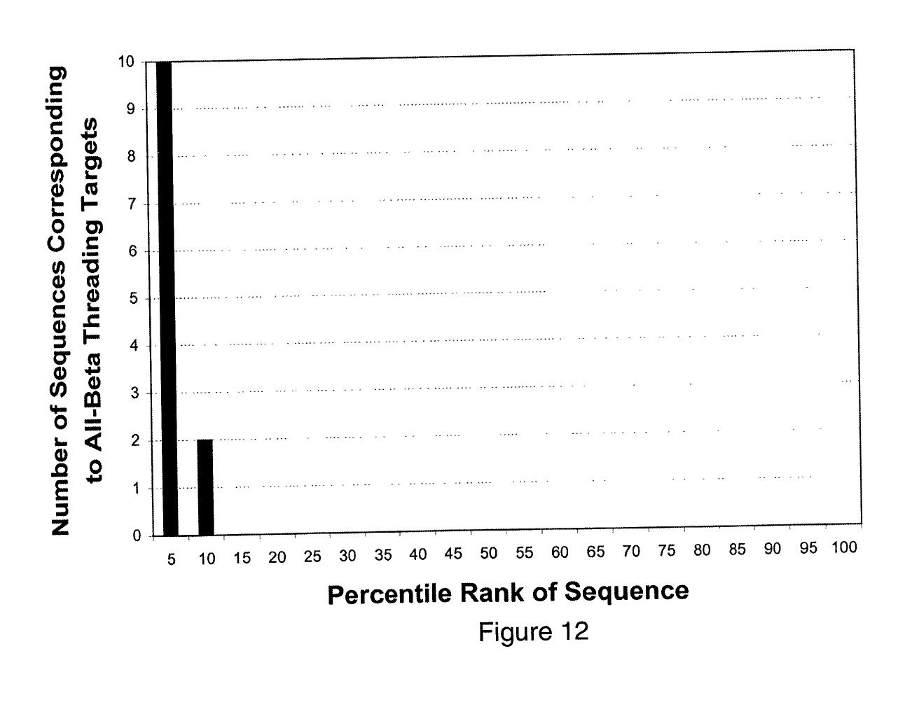

First, a scoring table was assembled from the 31 proteins in Table 5 that were classified by the SCOP database as being “All alpha” proteins. Second, the 12 “All beta” proteins from Table 5 were threaded using the scoring table derived solely from the “All alpha” proteins. In other words, amino acid propensities for the thermodynamic environments from all-alpha proteins were used to perform fold recognition experiments on all-beta proteins. For more than 80% of the targets (10/12), sequences known to adopt the native all-beta structures scored in the top 5% of the 3858 decoy sequences, (FIG. 12).

This result was a clear demonstration that the energetic information derived from the COREX calculations was independent of protein secondary structure.

### References

All patents and publications mentioned in the specification are indicative of the level of those skilled in the art to which the invention pertains. All patents and publications are herein incorporated by reference to the same extent as if each individual publication was specifically and individually indicated to be incorporated by reference.

Altschul et al., 1997, Nuc Acid Res 25: 3389-3402.

Anfinsen C B. 1973, Science 181: 223-230.

Bai & Englander, 1996, Proteins 24: 145-151.

Baker et al., 1992, Nature 356: 263-265.

Baldwin R L. 1986, Proc Natl Acad Sci USA 83: 8069-8072.

Bowie et al., 1991, Science 253: 164-170.

Chamberlain et al., 1996, Nat Struct Biol 3: 782-788.

Cohen F E. 1999, J Mol Biol 293: 313-320.

D'Aquino et al., 1996, Proteins 22: 404-412.

Feldman & Frydman J. 2000, Curr Opin Struct Biol 10: 26-33.

Fink A L. 1999, Physiol Rev 79: 425-449.

Gomez et al., 1995, Proteins 22: 404-412.

Gribskov et al., 1987, Proc Natl Acad Sci USA 84: 4355-4358.

Habermann & Murphy. 1996, Prot Sci 5: 1229-1239.

Hilser & Freire. 1996, J Mol Biol 262: 756-772.

Hilser et al., 1998, Proc Natl Acad Sci USA 95: 9903-9908.

Hobohm & Sander. 1994, Prot Sci 3: 522-524.

Huyghues-Despointes et al., 1999, Biochem 38: 16481-16490.

Jackson, 1998, Fold Des 3: R81-91.

Jaravine et al., 2000, Prot Sci 9: 290-301.

Jones et al., 1999. Proteins Suppl 3:104-111.

Kabsch & Sander. 1983. Biopolymers 22: 2577-2637.

Kuroda & Kim. 2000. J Mol Biol 298: 493-501.

Lee et al., 1994. Proteins 20: 68-84.

Llinas et al., 1999. Nat Struct Biol 6:1072-1078.

Murzin et al., 1995. J Mol Biol 247: 536-540.

Pan et al., 2000. Proc Natl Acad Sci USA 97:12020-12025.

Park et al., 1998. J Mol Biol 284: 1201-1210.

Pereira et al., 1999, Biophys. J. 76:2319-2328.

Pochapsky & Gopen. 1992. Protein Sci. 1:786-795.

Rice & Eisenberg. 1997. J Mol Biol 267: 1026-1038.

Sadqi et al., 1999. Biochem 38: 8899-8906.

Smith & Waterman. 1981. J Mol Biol 147: 195-197.

Swint-Kruse & Robertson. 1996. Biochem 35: 171-180.

Xie & Freire. 1994. J Mol Biol 242: 62-80.

Wrabl, et al., Protein Sci 10(5) 1032-45.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A protein database comprising nonhomologus proteins having known residue-specific free energies of folding.

2. The database of claim 1, wherein the nonhomologus proteins are globular proteins.

3. The database of claim 1, wherein the database is determined by a computational method comprising the step of determining a stability constant from the ratio of the summed probability of all states in the ensemble in which a residue j is in a folded conformation to the summed probability of all states in which j is in an unfolded conformation according to the equation,
$${K}_{f , j} = \frac{\sum {P}_{f , j}}{\sum {P}_{nf , j}} .$$

4. The database of claim 3, wherein the stability constants for the residues are arranged into at least one thermodynamic classification group selected from the group consisting of stability, enthalphy, and entropy.

5. The database of claim 4, wherein the stability classification group comprises high stability, medium stability or low stability.

6. The database of claim 5, wherein the residues in the high stability classification comprises phenylalanine, tryptophan or tyrosine.

7. The database of claim 5, wherein the residues in the low stability classification comprises glycine or proline.

8. The database of claim 5, wherein the residues in the medium stability classification comprises asparagine or glutamic acid.

9. The database of claim 4, wherein the enthalpy classification group comprises high enthalpy or low enthalpy.

10. The database of claim 4, wherein the entropy classification group comprises high entropy or low entropy.

11. The database of claim 3, wherein the stability constants for the residues are arranged into three thermodynamic classification groups selected from the group consisting of stability, enthalphy, and entropy.

12. The database of claim 3, wherein the stability constants for the residues are arranged into twelve thermodynamic classifications selected from the group consisting of HHH, LHH, HHL, MHL, LHL, HLL, MLL, LLL, HLH, MLH and LLH.

13. A method of developing a protein database comprising the steps of:
inputting high resolution structures of proteins;
generating an ensemble of incrementally different conformational states by combinatorial unfolding of a set of predefined folding units in all possible combinations of each protein;
determining the probability of each said conformational state;
calculating a residue-specific free energy of each said conformational state; and
classifying a stability constant into a thermodynamic classification group.

14. The method of claim 13, wherein the stability constant is arranged into at least one thermodynamic classification group selected from the group consisting of stability, enthalphy, and entropy.

15. The method of claim 13, wherein the protein database comprises nonhomologous proteins.

16. The method of claim 13, wherein the generating step comprises dividing the proteins into folding units by placing a block of windows over the entire sequence of the protein and sliding the block of windows one residue at a time.

17. The method of claim 13, wherein the determining step comprises determining the free energy of each of the conformational states in the ensemble; determining the Boltzmann weight [Kᵢ=exp(−ΔGᵢ/RT)] of each state; and determining the probability of each state using the equation
$${P}_{i} = \frac{{K}_{i}}{\sum {K}_{i}^{′}} .$$

18. The method of claim 13, wherein the calculating step comprises determining the energy difference between all microscopic states in which a particular residue is folded and all such states in which it is unfolded using the equation
ΔG_{f,j}=−RT·lnκ_{f,j}.

19. A method of identifying a protein fold comprising determining the distribution of amino acid residues in different thermodynamic environments corresponding to a known protein structure.

20. The method of claim 19, wherein the thermodynamic environments are selected from the group consisting of stability, enthalpy and entropy.

21. The method of claim 19, wherein determining the distribution of amino acid residues comprises constructing scoring matrices derived of thermodynamic information.

22. The method of claim 21, wherein the scoring matrices are derived from COREX stability, enthalpy or entropy information.

23. A system for developing a protein database and for identifying a protein fold comprising:
a protein database having a data structure for protein data, said data structure including data fields for thermodynamic classifications for amino acids of a protein; and
a computer-based program for identifying protein fold data for said database, said program having
an input module for receiving high resolution structure data for one or more proteins, and
a processing module for determining amino acid thermodynamic classifications for said one or more proteins and storing said amino acid thermodynamic classifications into said data fields of said protein database.

24. The system of claim 23, wherein said processing module is adapted for
generating an ensemble of incrementally different conformational state;
determination the probability of each said conformational state;
calculating a residue-specific free energy of each said conformational state; and
classifying a stability constant into a thermodynamic classification group.

25. The system of claim 24, wherein said computer-based program further includes a probability determination module for determining the free energy of each of the conformational states in the ensemble; determining the Boltzmann weight; and determining the probability of each state.

26. The system of claim 24, wherein said computer program further includes a display module for producing one or more graphical reports to a screen or a print-out.

27. The system of claim 26, wherein said one or more graphical reports is a display of a three-dimensional protein structure based on said amino acid thermodynamic classifications.

28. The system of claim 26, wherein said one or more graphical reports is a scatter-plot of normalized frequencies of COREX stability data versus normalized frequencies of average side chain surface exposure.

29. The system of claim 26, wherein said one or more graphical reports is a chart displaying thermodynamic environments for amino acids of a protein.

30. A computer-readable medium having computer-executable instructions for performing the steps recited in claim 13.

31. A computer-readable medium having computer-executable instructions for performing the steps recited in claim 16.

32. A computer-readable medium having computer-executable instructions for performing the steps recited in claim 17.

33. A computer-readable medium having computer-executable instructions for performing the steps recited in claim 18.

34. A computer-readable medium having computer-executable instructions for performing the steps recited in claim 19.

35. A computer-readable medium having computer-executable instructions for performing the steps recited in claim 22.

36. A database having a data structure which stores information defining thermodynamic classification groups, said database comprising:
a field for storing a value of an amino acid name or amino acid abbreviation; and
one or more classification fields for storing a value representing a numerical value for a thermodynamic classification for a particular amino acid.

37. A database according to claim 36, wherein said database further has a total field for storing a value representing the summed total of each of the numerical values for each thermodynamic classification for a particular amino acid.

38. The method of claim 13, wherein the protein database comprises globular proteins.
